(19) United States
(12) Patent Application Publication
(10) Pub. No.: US 2026/0021186 A1
(43) Pub. Date: Jan. 22, 2026

(54) Fatty Acid-D-Amino Acid Peptides Conjugates as Anaplerotic Compounds for Use in Treating Propionic Acidemia, Methylmalonic Acidurias, and Energy Metabolic Disorders

(71) Applicant: University of Pittsburgh - Of the Commonwealth System of Higher Education, Pittsburgh, PA (US)

(72) Inventor: Al-Walid A. Mohsen, Gibsonia, PA (US)

(21) Appl. No.: 18/997,209

(22) PCT Filed: Jul. 31, 2023
(86) PCT No.: PCT/US23/29101

§ 371 Date: Jan. 21, 2025

Related U.S. Application Data
(60) Provisional application No. 63/394,136, filed on Aug. 1, 2022.

(51) Int. Cl. A61K 47/54; A61K 47/64

(52) U.S. Cl. CPC A61K 47/542; A61K 47/64

(57) ABSTRACT

Provided herein is a method of providing an anaplerotic compound to a patient in need therefor. The method may be used to treat the patient for a propionic acidemia. a methymalonic aciduria, or a fatty acid oxidation disorder. Also provided herein are compounds useful in those methods. the compounds comprising an adipic acid or methylmalonic acid moiety linked to from one to three amino acids, e.g. D-amino acids, such as D-Ser, D-His, D-Lys, D-Leu, and/or D-Ala.

### CROSS REFERENCE TO RELATED APPLICATION

This application claims priority to U.S. Provisional Patent Application No. 63/394,136 filed Aug. 1, 2022, the disclosure of which is incorporated herein by reference in its entirety.

### STATEMENT REGARDING FEDERAL FUNDING

This invention was made with government support under Grant Nos. DK054936 and DK109907-05A1, awarded by the National Institutes of Health. The government has certain rights in the invention.

Provided herein are methods of treating propionic acidemia and methylmalonic acidurias, glutaric acidemia I and glutaric acidemia II, and fatty acid oxidation disorders. Also provided herein are compounds, composition of matter, specifically designed for treating propionic acidemia and methylmalonic acidurias and fatty acid oxidation disorders.

Disease conditions that result from deficiency of succinyl-CoA include propionic acidemia (PA) (OMIM 606054); inborn errors of metabolism causing methylmalonic acidurias include those described in connection with OMIM 21000 (Bchr Syndrome), OMIM 609058 (methylmalonyl-CoA mutase), OMIM 613646 (methylmalonic aciduria, transient, due to transcobalamin receptor defect), OMIM 251100 (methylmalonic aciduria, cblA type), OMIM 251110 (methylmalonic aciduria, cblB type); glutaric acidemia I (GA1) OMIM 231670 and glutaric acidemia II or glutaric aciduria II (GA2) OMIM 231680; and fatty acid oxidation disorders include medium chain acyl-CoA dehydrogenase (MCAD) deficiency (OMIM 201450); very long chain acyl-CoA dehydrogenase (VLCAD) deficiency (OMIM 201475); trifunctional protein (TFP) deficiency (OMIM 609015); long chain 3-hydroxyacyl-CoA dehydrogenase (LCHAD) deficiency (OMIM 609016); and carnitine palmitoyl transferase II (CPT II) deficiency (OMIM 600649). Also included is any disorder that causes mitochondrial dysfunction that results in reduction in mitochondrial ATP production.

Anaplerosis is the re-filling of the catalytic intermediates of the cycle that carry acetyl-CoA as it is oxidized. Major anaplerotic cellular substrates include pyruvate, glutamine/glutamate, aspartate/asparagine, and precursors of propionyl-CoA (odd-chain fatty acids, specific amino acids, C (5)-ketone bodies). Compounds, compositions, and treatment methods useful in the replenishment of deficiencies in TCA cycle intermediates, such as succinyl-CoA deficiencies, are desirable.

### SUMMARY

Provided herein according to one aspect or embodiment is a method of providing an anaplerotic compound to a patient in need therefor, comprising administering to the patient a compound having the structure A-B or B-A′ where: A comprises a dicarboxylate, a tricarboxylic, or a straight or branched-chain fatty acid moiety selected from: an adipic acid, a 3-hydroxy-adipic acid, a 3-ketoadipic acid, a glutaric acid, a succinic acid, methylmalonic acid, a citric acid, an isocitric acid, α-ketoglutaric acid, an oxaloacetic acid, a heptanoic acid, a 2,6-dimethylheptanoic acid, a 4,8-dimethylnonanoic acid, a 6-amino-2,4-dimethylheptanoic acid, a straight chain fatty acid, or a branched-chain fatty acid; A′ comprises a straight or branched-chain fatty acid moiety selected from: 6-hydroxy-hexanoic acid (hydroxyl linked to B carboxylate as an ester bond), 6-amino-hexanoic acid (linked to B carboxylate through an amide bond), or 6-amino-2,4-dimethylheptanoic acid (linked to B carboxylate through an amide bond); and B is an amino acid, D-dipeptide, or D-tripeptide of D-amino acids, comprising at least one amino acid selected from D-Ser, D-Ala, D-His, D-Lys, D-Glu, D-Gln, D-Asp, D-Asn, D-Leu, D-Ile, D-Thr, or D-Val linked to A by an amide bond or by an amide bond or an ester bond in the case of D-Ser or linked to A′ by an ester bond or an amide bond, or a pharmaceutically acceptable salt thereof.

Also provided herein according to one aspect or embodiment is a method of treating a patient having a propionic acidemia (PA), a methymalonic aciduria (MMA), or a fatty acid oxidation disorder, and having abnormally low amounts of anaplerotic intermediates, comprising administering to the patient a compound having the structure A-B or B-A′, wherein:
a. for treating PA, A comprises a dicarboxylate, a tricarboxylic, or an even straight-chain fatty acid moiety selected from: an adipic acid, a glutaric acid, a succinic acid, methylmalonic acid, a citric acid, an isocitric acid, an oxaloacetic acid, α-ketoglutaric acid, A′ comprises a straight even-chain dicarboxylic or fatty acid moiety selected from: a 6-hydroxy-hexanoic acid, a 6-amino-hexanoic acid, and B is a D-amino acid, D-dipeptide, or D-tripeptide of D-amino acids comprising at least one amino acid selected from D-Ser, D-Ala, D-His, D-Lys, D-Glu, D-Gln, D-Asp, D-Asn, or D-Leu linked to A by an amide bond or by an amide bond or ester bond in the case of D-Ser or to A′ by an amide bond or an ester bond, or a pharmaceutically acceptable salt thereof;
b. for treating MMA, A comprises a dicarboxylate, a tricarboxylic, or an even straight-chain fatty acid moiety selected from: an adipic acid, a glutaric acid, a succinic acid, a citric acid, an isocitric acid, an oxaloacetic acid, and α-ketoglutaric acid; and A′ comprises a straight even-chain dicarboxylic or fatty acid moicty selected from: 6-hydroxy-hexanoic acid or 6-amino-hexanoic acid; and B is a D-amino acid, D-dipeptide, or D-tripeptide of D-amino acids, comprising at least one amino acid selected from D-Ser, D-Ala, D-His, D-Lys, D-Glu, D-Gln, D-Asp, D-Asn, or D-Leu linked to A by an amide bond or by an amide bond or ester bond in the case of D-Ser or to A′ by an amide bond or an ester bond, or a pharmaceutically acceptable salt thereof; or
c. for treating a fatty acid oxidation disorder, A comprises a dicarboxylate, a tricarboxylic, or an even straight-chain or branched-chain fatty acid moiety selected from: an adipic acid, a glutaric acid, a succinic acid, methylmalonic acid, citric acid, an isocitric acid, an oxaloacetic acid, a ketoglutaric acid, a heptanoic acid, a 2,6-dimethylheptanoic acid, a 4,8-dimethylnonanoic acid, a 6-amino-2,4-dimethylheptanoic acid, an even straight chain fatty acid, or a branched-chain fatty acid; A′ comprises a straight even-chain dicarboxylic or fatty acid moicty selected from: 6-hydroxy-hexanoic acid, 6-amino-hexanoic acid, or 6-amino-2,4-dimethylheptanoic acid; and B is a D-amino acid, D-dipeptide, or D-tripeptide of D-amino acids, comprising at least one amino acid selected from D-Ser, D-Ala, D-His, D-Lys, D-Glu, D-Gln, D-Thr, D-Ile, D-Val, D-Asp, D-Asn, or D-Leu linked to A by an amide bond or by an amide bond or ester bond in the case of D-Ser or to A′ by an amide bond or an ester bond, or a pharmaceutically acceptable salt thereof.

According to a further aspect or embodiment, also provided herein is a compound having the structure A-B or B-A′, wherein A comprises a dicarboxylate, a tricarboxylic, or a straight or branched-chain fatty acid moiety selected from: an adipic acid, a 3-hydroxy-adipic acid, a 3-ketoadipic acid, a glutaric acid, a succinic acid, methylmalonic acid, a citric acid, an isocitric acid, α-ketoglutaric acid, an oxaloacetic acid, a heptanoic acid, a 2,6-dimethylheptanoic acid, a 4,8-dimethylnonanoic acid, a 6-amino-2,4-dimethylheptanoic acid, a straight chain fatty acid, or a branched-chain fatty acid; A′ comprises a straight or branched-chain fatty acid moiety selected from: 6-hydroxy-hexanoic acid (hydroxyl linked to B carboxylate as an ester bond), 6-amino-hexanoic acid (linked to B carboxylate through an amide bond), or 6-amino-2,4-dimethylheptanoic acid (linked to B carboxylate through an amide bond); and B is an amino acid, D-dipeptide, or D-tripeptide of amino acids, comprising at least one amino acid selected from D-Ser, D-Ala, D-His, D-Lys, D-Glu, D-Gln, D-Asp, D-Asn, D-Leu, D-Ile, D-Thr, or D-Val linked to A by an amide bond or by an amide bond or ester bond in the case of D-Ser, or to A′ by an ester bond or an amide bond, or a pharmaceutically acceptable salt thereof.

The following numbered clauses outline various aspects or embodiments of the present invention:
Clause 1. A method of providing an anaplerotic compound to a patient in need therefor, comprising administering to the patient a compound having the structure A-B or B-A′, where: A comprises a dicarboxylate, a tricarboxylic, or a straight or branched-chain fatty acid moiety selected from: an adipic acid, a 3-hydroxy-adipic acid, a 3-ketoadipic acid, a glutaric acid, a succinic acid, methylmalonic acid, a citric acid, an isocitric acid, α-ketoglutaric acid, an oxaloacetic acid, a heptanoic acid, a 2,6-dimethylheptanoic acid, a 4,8-dimethylnonanoic acid, a 6-amino-2,4-dimethylheptanoic acid, a straight chain fatty acid, or a branched-chain fatty acid; A′ comprises a straight or branched-chain fatty acid moiety selected from: 6-hydroxy-hexanoic acid (hydroxyl linked to B carboxylate as an ester bond), 6-amino-hexanoic acid (linked to B carboxylate through an amide bond), or 6-amino-2,4-dimethylheptanoic acid (linked to B carboxylate through an amide bond); and B is an amino acid, D-dipeptide, or D-tripeptide of from D-amino acids, comprising at least one amino acid selected from D-Ser, D-Ala, D-His, D-Lys, D-Glu, D-Gln, D-Asp, D-Asn, D-Leu, D-Ile, D-Thr, or D-Val linked to A by an amide bond or by an amide bond or ester bond in the case of D-Ser, or to A′by an ester bond or an amide bond, or a pharmaceutically acceptable salt thereof.
Clause 2. The method of clause 1, wherein the compound has the structure A-B. where: A comprises an adipic acid moiety or a methylmalonic acid moiety; and B comprises 1-3 of D-amino acids selected from D-Ser, D-Ala, D-Lys, D-Leu, or D-His linked to A by an amide bond or by an amide bond or an ester bond in the case of D-Ser, or a pharmaceutically acceptable salt thereof for the treatment of fatty acid oxidation disorders.
Clause 3. The method of clause 1 or 2, wherein the generated anaplerotic products enter the Krebs cycle at various points as intermediates, such as, for example, acetyl-CoA, citrate, isocitrate, α-ketoglutarate, succinyl-CoA, and/or oxaloacetate.
Clause 4. A method of treating a patient having a propionic acidemia (PA), a methymalonic aciduria (MMA), or a fatty acid oxidation disorder, and having abnormally low amounts of anaplerotic intermediates, comprising administering to the patient a compound having the structure A-B or B-A′, wherein:
   a. for treating PA, A comprises a dicarboxylate, a tricarboxylic, or an even straight-chain fatty acid moiety selected from: an adipic acid, a glutaric acid, a succinic acid, methylmalonic acid, a citric acid, an isocitric acid, an oxaloacetic acid, α-ketoglutaric acid, A′ comprises a straight even-chain dicarboxylic or fatty acid moiety selected from: a 6-hydroxy-hexanoic acid, a 6-amino-hexanoic acid, and B is a D-amino acid, D-dipeptide, or D-tripeptide of D-amino acids comprising at least one amino acid selected from D-Ser, D-Ala, D-His, D-Lys, D-Glu, D-Gln, D-Asp, D-Asn, or D-Leu linked to A by an amide bond or by an amide bond or ester bond in the case of D-Ser or to A′ by an amide bond or an ester bond, or a pharmaceutically acceptable salt thereof;
   b. for treating MMA, A comprises a dicarboxylate, a tricarboxylic, or an even straight-chain fatty acid moiety selected from: an adipic acid, a glutaric acid, a succinic acid, a citric acid, an isocitric acid, an oxaloacetic acid, and α-ketoglutaric acid; and A′ comprises a straight even-chain dicarboxylic or fatty acid moiety selected from: a 6-hydroxy-hexanoic acid or a 6-amino-hexanoic acid; and B is a D-amino acid, D-dipeptide, or D-tripeptide of D-amino acids, comprising at least one amino acid selected from D-Ser, D-Ala, D-His, D-Lys, D-Glu, D-Gln, D-Asp, D-Asn, or D-Leu linked to A by an amide bond or by an amide bond or ester bond in the case of D-Ser or to A′ by an amide bond or an ester bond, or a pharmaceutically acceptable salt thereof; or
   c. for treating a fatty acid oxidation disorder, A comprises a dicarboxylate, a tricarboxylic, or an even straight-chain or branched-chain fatty acid moiety selected from: an adipic acid, a glutaric acid, a succinic acid, methylmalonic acid, a citric acid, an isocitric acid, an oxaloacetic acid, α-ketoglutaric acid, a heptanoic acid, a 2,6-dimethylheptanoic acid, a 4,8-dimethylnonanoic acid, a 6-amino-2,4-dimethylheptanoic acid, an even straight chain fatty acid, or a branched-chain fatty acid; A′ comprises a straight even-chain dicarboxylic or fatty acid moiety selected from: 6-hydroxy-hexanoic acid, 6-amino-hexanoic acid, or 6-amino-2,4-dimethylheptanoic acid; and B is a D-amino acid, D-dipeptide, or D-tripeptide of D-amino acids, comprising at least one amino acid selected from D-Ser, D-Ala, D-His, D-Lys, D-Glu, D-Gln, D-Thr, D-Ile, D-Val, D-Asp, D-Asn, or D-Leu linked to A by an amide bond or by an amide bond or ester bond in the case of D-Ser or to A′ by an amide bond or an ester bond, or a pharmaceutically acceptable salt thereof.
Clause 5. The method of clause 4, wherein the compound has the structure A-B. where: A comprises an adipic acid moicty or a methylmalonic acid moiety (not for MMA); and B is a D-amino acid, D-dipeptide, or D-tripeptide of D-amino acids, comprising at least one amino acid selected from D-Ser, D-Ala, D-Lys, D-Leu, and D-His, linked to A by an amide bond or by an amide bond or an ester bond in the case of D-Ser, or a pharmaceutically acceptable salt thereof, in an amount effective to normalize levels of the anaplerotic intermediates.
Clause 6. The method of any one of clauses 1-5, wherein the patient has propionic acidemia (PA); methylmalonic aciduria (MMA) transient, due to transcobalamin receptor defect; methylmalonic aciduria, cblA type; methylmalonic aciduria, cblB type; medium chain acyl-CoA dehydrogenase (MCAD) deficiency; very long chain acyl-CoA dehydrogenase (VLCAD) deficiency; trifunctional protein (TFP) deficiency; long chain 3-hydroxyacyl-CoA dehydrogenase (LCHAD) deficiency; carnitine palmitoyl transferase II (CPT II) deficiency; glutaric acidemia I (GA I or GA1); or glutaric acidemia II (GA I or GA2).
Clause 7. The method of any one of clauses 1-5, wherein the compound comprises a moicty type listed in FIGS. 17A and 17B and the compound is used for treatment of a corresponding recommended indication in FIGS. 17A and 17B, and, where relevant, is optionally contraindicated for treatment of a corresponding disease as indicated in FIGS. 17A and 17B.
Clause 8. The method of any one of clauses 1-7, wherein the patient has reduced succinyl-CoA or lysine succinylation.
Clause 9. The method of any one of clauses 1-5, wherein the patient has glutaric acidemia I (GA I or GA1) with the exclusion of glutaric acid or lysine in the compound structure, or glutaric acidemia II (GA 2 or GA II) with the exclusion of glutaric acid or lysine, fatty acids and branched chain amino acids that require ETF or ETF dehydrogenase for their breakdown in the compound structure. GA II cannot have: Ile, Leu, Val, Lys, glutaric acid or adipic acid, branched-chain or odd chain fatty acids fatty acids, or any saturated fatty acid, C4 and higher.
Clause 10. The method of any one of clauses 1-9, wherein B comprises one or more of D-Ser, D-Ala, D-Lys, D-Leu, and/or D-His.
Clause 11. The method of any one of clauses 1-10, wherein B is chosen from: -D-Ser, -D-Ala, -D-His, -D-His-D-Ala-D-Ser, -D-His-D-Ser-D-Ala, -D-Ser-D-His-D-Ala, -D-Ser-D-Ala-D-His, -D-Ala-D-Ser-D-His, -D-Ala-D-His-D-Ser, -D-Ala-D-His-D-Ser, -D-His-D-Ala, -D-His-D-Ser, -D-Ser-D-His, -D-Ala-D-Ser, or -D-Ala-D-His, wherein D-Ser is linked by an amide bond (—N-linked) or an ester bond (—O-linked).
Clause 12. The method of any one of clauses 1-9, the compound having the

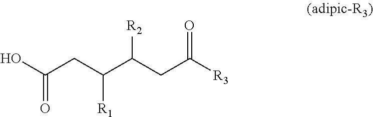

where R₁ and R₂ are, independently H, hydroxyl, or oxygen connected by a double bond to form a keto moiety, where at least one of R₁ and R₂ is H, and R₃ is linked via an ester bond (—O-linked) or amide (—N-linked) bond, and comprises from 1-3 D amino acids selected from D-Ser, D-Ala, D-His, D-Lys, D-Glu, D-Gln, D-Asp, D-Asn, D-Leu, and D-Val, or is one of: -D-Ser, —O-D-Ser, -D-Ala, -D-His, -D-His-D-Ala-D-Ser, -D-His-D-Ala-O-D-Ser, -D-His-D-Ser-D-Ala, -D-His-O-D-Ser-D-Ala, -D-Ser-D-His-D-Ala, —O-D-Ser-D-His-D-Ala, -D-Ser-D-Ala-D-His, —O-D-Ser-D-Ala-D-His, -D-Ala-D-Ser-D-His, -D-Ala-O-D-Ser-D-His, -D-Ala-D-His-D-Ser, -D-Ala-D-His-D-Ser, -D-His-D-Ala, -D-His-D-Ser, -D-His-O-D-Ser, -D-Ser-D-His, -D-Ala-O-D-Ser, or -D-Ala-D-His.
Clause 13. The method of any one of clauses 1-9, the compound having the structure:

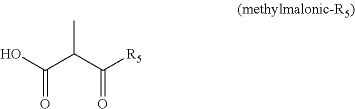

where R₅ is linked via an ester (—O-linked) or amide (—N-linked) bond, and comprises from 1-3 D amino acids selected from D-Ser, D-Ala, D-His, D-Lys, D-Glu, D-Gln, D-Asp, D-Asn, D-Leu, and D-Val, or is one of: -D-Ser, —O-D-Ser, -D-Ala, -D-His, -D-His-D-Ala-D-Ser, -D-His-D-Ala-O-D-Ser, -D-His-D-Ser-D-Ala, -D-His-O-D-Ser-D-Ala, -D-Ser-D-His-D-Ala, —O-D-Ser-D-His-D-Ala, -D-Ser-D-Ala-D-His, —O-D-Ser-D-Ala-D-His, —D-Ala-D-Ser-D-His, -D-Ala-O-D-Ser-D-His, -D-Ala-D-His-D-Ser, -D-Ala-D-His-D-Ser, -D-His-D-Ala, -D-His-D-Ser, -D-His-O-D-Ser, -D-Ser-D-His, —O-D-Ser-D-His-D-Ser-D-Ala, —O-D-Ser-D-Ala, -D-Ala-D-Ser, -D-Ala-O-D-Ser, -D-Ala-D-His, -D-Leu-D-His, or -D-Lys-D-His.
Clause 14. The method of any one of clauses 1-9, wherein the compound is chosen from: PMA001, e.g., 4-hydroxyladipic acid-O-D-Ser; PMA002, e.g., 4-hydroxyladipic acid-O-D-Ser-D-Ala; PMA003, e.g., 4-hydroxyladipic acid-O-D-Ser-D-Ala; PMA004, e.g., 2-ketoglutaric acid-D-2-hydroxyglycine-D-Ala; PMA005, e.g., 2-ketoglutaric acid-O-D-Ser-D-Ala; PMA006, e.g., 4-ketoadipic acid-O-L-Ser; PMA007.1, e.g., 4-ketoadipic acid-O-D-Ser-D-His; PMA008, e.g., 2-ketoglutaric acid-O-D-Ser-D-His; PMA009, e.g., 2-ketoglutaric acid-D-Ala-D-His; PMA010.1, e.g., 4-ketoadipic acid-D-Ala-D-His; PMA011, e.g., methylmalonic acid-O-D-Ser-D-His; PMA012, e.g., methylmalonic acid-D-Ala-D-His; PMA013, e.g., adipic acid-D-Ala-D-Ser-D-His; PMA014, e.g., adipic acid-D-Ser-D-His; PMA019, e.g., methylmalonic acid-D-Leu-D-His; and PMA020, e.g., methylmalonic acid-D-Lys-D-His.
Clause 15. The method of any one of clauses 1-9, wherein the compound is PMA010 (adipic-D-Ala-D-His).
Clause 16. The method of any one of clauses 1-9, wherein the compound is PMA007 (adipic-O-D-Ser-D-His).
Clause 17. The method of any one of clauses 1-9, wherein the compound is PMA011 (methylmalonic-D-Ala-D-His), not for MMA.
Clause 18. The method of any one of clauses 1-9, wherein the compound is PMA019 (methylmalonic-D-Leu-D-His), not for MMA.
Clause 19. The method of any one of clauses 1-9, wherein the compound is PMA020 (methylmalonic-D-Lys-D-His), not for MMA.
Clause 20. The method of any one of clauses 1-11, the patient having a propionic acidemia, and where A is methylmalonic acid, excluding propiogenic amino acids or fatty acids.
Clause 21. The method of clause 20, wherein the patient does not have a methylmalonic aciduria, excluding methylmalonic acid.
Clause 22. The method of any one of clauses 1-21, wherein the patient has propionic acidemia or methylmalonic aciduria, and the method further comprises administering to the patient an inhibitor that blocks the formation of the propionoyl-CoA or methylmalonoyl-CoA.
Clause 23. The method of clause 22, wherein the inhibitor that blocks the formation of the propionoyl-CoA acid or methylmalonoyl-CoA is 2,2-dimethybutyric acid or sodium 2,2-dimethylbutanoate.
Clause 24. The method of any one of clauses 1-21, further comprising administering to the patient, e.g., coadministering to the patient as an adjuvant therapy, an inhibitor that restricts propionyl-CoA or methylmalonyl-CoA production from various sources.
Clause 25. The method of clause 24, wherein the inhibitor is an inhibitor of propionyl-CoA or methylmalonyl-CoA formation, such as HST5040.
Clause 26. A compound having the structure A-B or B-A′, wherein A comprises a dicarboxylate, a tricarboxylic, or a straight or branched-chain fatty acid moiety selected from: an adipic acid, a 3-hydroxy-adipic acid, a 3-ketoadipic acid, a glutaric acid, a succinic acid, methylmalonic acid, a citric acid, an isocitric acid, α-ketoglutaric acid, an oxaloacetic acid, a heptanoic acid, a 2,6-dimethylheptanoic acid, a 4,8-dimethylnonanoic acid, a 6-amino-2,4-dimethylheptanoic acid, a straight chain fatty acid, or a branched-chain fatty acid; A′ comprises a straight or branched-chain fatty acid moiety selected from: 6-hydroxy-hexanoic acid (hydroxyl linked to B carboxylate as an ester bond), 6-amino-hexanoic acid (linked to B carboxylate through an amide bond), or 6-amino-2,4-dimethylheptanoic acid (linked to B carboxylate through an amide bond); and B is an amino acid, D-dipeptide, or D-tripeptide of amino acids, comprising at least one amino acid selected from D-Ser, D-Ala, D-His, D-Lys, D-Glu, D-Gln, D-Asp, D-Asn, D-Leu, D-Ile, D-Thr, or D-Val linked to A by an amide bond or by an amide bond or ester bond in the case of D-Ser, or to A′ by an ester bond or an amide bond, or a pharmaceutically acceptable salt thereof.
Clause 27. The compound of clause 26, wherein the compound has the structure A-B, where: A comprises an adipic acid moiety or a methylmalonic acid moiety; and B is a D-amino acid, D-dipeptide, or D-tripeptide of D-amino acids, comprising at least one amino acid selected from D-Ser, D-Ala, D-Lys, D-Leu, and D-His, linked to A by an amide bond or by an amide bond or an ester bond in the case of D-Ser, or a pharmaceutically acceptable salt thereof.
Clause 28. The compound of clause 26, wherein B comprises one or more of D-Ser, D-Ala, D-Lys, D-Leu, and/or D-His.
Clause 29. The compound of clause 28, wherein B is chosen from: -D-Ser, -D-Ala, -D-His, -D-His-D-Ala-D-Ser, -D-His-D-Ser-D-Ala, -D-Ser-D-His-D-Ala, -D-Ser-D-Ala-D-His, -D-Ala-D-Ser-D-His, -D-Ala-D-His-D-Ser, -D-Ala-D-His-D-Ser, -D-His-D-Ala,-D-His-D-Ser, -D-Ser-D-His, -D-Ala-D-Ser, or -D-Ala-D-His, wherein D-Ser is linked by an amide bond (—N-linked) or an ester bond (—O-linked).
Clause 30. The compound of clause 26, having the structure:

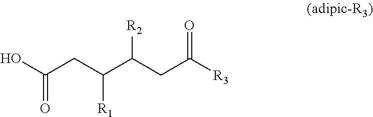

where R₁ and R₂ are, independently H, hydroxyl, or oxygen connected by a double bond to form a keto moiety, where at least one of R₁ and R₂ is H, and R₃ is linked via an ester (—O-linked) or amide (—N-linked) bond, and comprises from 1-3 D amino acids selected from D-Ser, D-Ala, D-His, D-Lys, D-Glu, D-Gln, D-Asp, D-Asn, D-Leu, and D-Val or is one of: -D-Ser, —O-D-Ser, -D-Ala,-D-His, -D-His-D-Ala-D-Ser, -D-His-D-Ala-O-D-Ser, -D-His-D-Ser-D-Ala, -D-His-O-D-Ser-D-Ala, -D-Ser-D-His-D-Ala, —O-D-Ser-D-His-D-Ala, -D-Ser-D-Ala-D-His, —O-D-Ser-D-Ala-D-His, -D-Ala-D-Ser-D-His, -D-Ala-O-D-Ser-D-His, -D-Ala-D-His-D-Ser, -D-Ala-D-His-D-Ser, -D-His-D-Ala, -D-His-D-Ser, -D-His-O-D-Ser, -D-Ser-D-His, -D-Ala-O-D-Ser, or -D-Ala-D-His.
Clause 31. The compound of clause 26, having the structure:

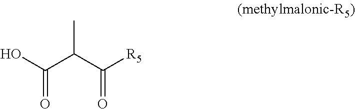

where R₅ is linked via an ester (—O-linked) or amide (—N-linked) bond, and comprises from 1-3 D amino acids selected from D-Ser, —O-D-Ser, D-Ala, D-His, D-Lys, D-Glu, D-Gln, D-Asp, D-Asn, D-Leu, and D-Val, or is one of: -D-Ser, —O-D-Ser, -D-Ala, -D-His, -D-His-D-Ala-D-Ser, -D-His-D-Ala-O-D-Ser, -D-His-D-Ser-D-Ala, -D-His-O-D-Ser-D-Ala, -D-Ser-D-His-D-Ala, —O-D-Ser-D-His-D-Ala, -D-Ser-D-Ala-D-His, —O-D-Ser-D-Ala-D-His, -D-Ala-D-Ser-D-His, -D-Ala-O-D-Ser-D-His, -D-Ala-D-His-D-Ser, -D-Ala-D-His-D-Ser, -D-His-D-Ala, -D-His-D-Ser, -D-His-O-D-Ser, -D-Ser-D-His, —O-D-Ser-D-His-D-Ser-D-Ala, —O-D-Ser-D-Ala-D-Ala-D-Ser, -D-Ala-O-D-Ser, -D-Ala-D-His, -D-Leu-D-His, or -D-Lys-D-His.
Clause 32. The compound of clause 25, chosen from: PMA001, e.g., 4-hydroxyladipic acid-O-D-Ser; PMA002, e.g., 4-hydroxyladipic acid-O-D-Ser-D-Ala; PMA003, e.g., 4-hydroxyladipic acid-O-D-Ser-D-Ala; PMA004, e.g., 2-ketoglutaric acid-D-2-hydroxyglycine-D-Ala; PMA005, e.g., 2-ketoglutaric acid-O-D-Ser-D-Ala; PMA006, e.g., 4-ketoadipic acid-O-L-Ser; PMA007.1, e.g., 4-ketoadipic acid-O-D-Ser-D-His-succinyl CoA; PMA008, e.g., 2-ketoglutaric acid-O-D-Ser-D-His; PMA009, e.g., 2-ketoglutaric acid-D-Ala-D-His; PMA010.1, e.g., 4-ketoadipic acid-D-Ala-D-His; PMA011, e.g., methylmalonic acid-O-D-Ser-D-His; PMA012, e.g., methylmalonic acid-D-Ala-D-His; and PMA013, e.g., adipic acid-D-Ala-D-Ser-D-His; PMA014, e.g., adipic acid-D-Ser-D-His; PMA019, e.g., methylmalonic acid-D-Leu-D-His; and PMA020, e.g., methylmalonic acid-D-Lys-D-His.

### BRIEF DESCRIPTION OF THE DRAWINGS

FIGS. 1A-1C provide exemplary compounds as described herein where the fatty acyl moiety and D-amino acid, dipeptide, or the oligopeptide moicty are varied. FIG. 1A provides exemplary compounds of dicarboxylates linked to a D-amino acid through an ester bond provided by the hydroxyl oxygen of D-Serine and where the dicarboxylate is a hydroxyl or keto substituted dicarboxylate. FIG. 1B provides exemplary compounds of dicarboxylates linked to a D-amino acid through an amide bond. FIG. 1C provides exemplary compounds of fatty acids (R) linked to a D-amino acid, dipeptide, or oligopeptide through an amide bond, the compounds of which are suitable for treating pyruvate dehydrogenase deficiency. In FIG. 1C, in PMA015.x, PMA016.x, PMA017.x, PMA018.x, PMA019.x, and PMA020.x, x can be an integer from 1-8, where: when x is 1, R is adipyl; when x is 2, R is glutaryl; when x is 3, R is succinyl; when x is 4, R is methylmalonyl; when x is 5, R is heptanoyl; when x is 6, R is 2,6-dimethylheptanoyl; when x is 7, R is 4,8-dimethylheptanoyl; and when x is 8, R is 6-amino-2,4-dimethylheptanoyl.

FIG. 2 is a schematic of the Krebs cycle, a.k.a., TCA or Citric acid cycle showing disorders causing depletion of intermediates of the cycle. Amino acids with significant contribution as anaplerotic are depicted. Disorders mentioned abbreviations: FAODs, Fatty Acids Oxidation Disorders; MADD, Multiple Acyl-CoA Dehydrogenases Deficiency (glutaric aciduria II, GA2, caused by ETF or ETF dehydrogenase deficiency); PA, Propionic Acidemia; MMA, Methylmalonic Aciduria; PDHD, Pyruvate Dehydrogenase Deficiency; SUCLA2D, Succinyl-CoA synthetase (SUCLA2) deficiency; FD, Fumarase Deficiency; MDH2, mitochondrial Malate Dehydrogenase Deficiency.

FIG. 3 is a propionyl-CoA metabolic pathway schematic illustrating its role as a major source of succinyl-CoA and the role of succinyl-CoA in cell physiology. Propiogenic amino acids has been estimated to account for 46%±21 of the total propionic acid output in PA patients. Propionic acid produced by gut bacteria was estimated to account for 22%±9 in blood circulation and ˜30% was attributed fatty acid sources. The physiological role of succinyl-CoA is intricate with its role in lysine succinylation of hundreds of mitochondrial and cellular proteins, and hence their function, dramatically affected by its depletion in PA patients and other metabolic disorders.

FIG. 4 shows that five known enzymes catalyze the first step of the pathway with overlapping substrate chain length specificity. The rest of the pathway reactions are carried out by the Trifunctional Protein heterooctamer, TFP, which carries out three different reactions to complete the cycle. The cycle output products include acetyl-CoA, which feeds into the TCA cycle, and propionyl-CoA that is converted to succinyl-CoA.

FIG. 5 depicts structures of the compounds coded PMA007 (adipic acid-O-D-Ser-D-His; 6-((R)-2-amino-3-(((S)-1-carboxy-2-(1H-imidazol-4-yl)ethyl)amino)-3-oxopropoxy)-6-oxohexanoic acid as described herein) and PMA010 (adipic acid-D-Ala-D-His; 6-(((R)-1-(((R)-1-carboxy-2-(1H-imidazol-4-yl)ethyl)amino)-1-oxopropan-2-yl)amino)-6 -oxohexanoic acid as described herein).

FIG. 6 depicts the structures of the compounds coded PMA011 (methylmalonic acid-D-Ala-D-His; 3-(((R)-1-(((R)-1-carboxy-2-(1H-imidazol-4-yl)ethyl)amino)-1-oxopropan-2-yl)amino)-2-methyl-3-oxopropanoic acid as described herein); PMA019 (methylmalonic acid-D-Leu-D-His; 3-(((R)-1-(((R)-1-carboxy-2-(1H-imidazol-4-yl)ethyl)amino)-4-methyl-1-oxopentan-2-yl)amino)-2-methyl-3-oxopropanoate as described herein); and PMA020 (methylmalonic acid-D-Lys-D-His; 3-(((R)-6-amino-1-(((R)-1-carboxy-2-(1H-imidazol-4-yl)ethyl)amino)-1-oxohexan-2-yl)amino)-2-methyl-3-oxopropanoate as described herein).

FIG. 7 are immunocytochemistry photomicrographs showing the effect of incubation of Fb859 propionic acidemia patient fibroblasts on for lysine succinylation with 90 μM of PMA010, 90 μM of PMA011, 90 μM of succinic acid, 90 μM of methylmalonic acid, or 90 μM adipic acid for 72 hours. Immunocytochemistry of the fibroblast cells were stained for anti-succinyllysine antibody, anti-MTCO1 antibody and DAPI for nuclei. Instrument: Zeiss LSM 710 confocal Microscopy. Magnification: 40×. The cells were grown in glass coverslips at a seeding density of 3-5×10⁴ cells. The media for untreated and treatment groups had no glucose, glutamine or pyruvate, and lipid stripped FBS was included instead of regular FBS. Scale bar=20 μm.

FIGS. 8A-8F are a comparison of lysine succinylation of cellular proteins visualized by immunofluorescence confocal microscopy in a Propionic Acidemia (Fb859) patient untreated (FIG. 8A) and treated with 30 μM and 90 μM of D-Serine (FIG. 8B), 30 M and 90 μM of PMA007 (FIG. 8C), or 30 μM and 90 μM of PMA010 (FIG. 8D). Scale bar=20 μm. FIG. 8E is a bar graph showing the quantification of the percent change in lysine succinylation (green) signal, from the immunofluorescence confocal micrographs of FIGS. 8B and 8C, in response to the treatment of D-serine and PMA007. FIG. 8F is a bar graph showing the quantification of the percent change in lysine succinylation (green) signal, from the immunofluorescence confocal micrographs of a D-Serine repeat experiment and FIG. 8D, in response to the treatment of D-serine and PMA010. Asterisks indicate statistical significance calculated at the raw data level (n=3); **P<0.01, ***P<0.001, ****P<0.0001 statistically compared to untreated. Statistical analysis used T-test.

FIGS. 9A-9F are a comparison of lysine succinylation of cellular proteins visualized by immunofluorescence confocal microscopy in human HEK293 control and HEK293 PCCA^{-/-} cells untreated (FIGS. 9A and 9B) and treated with 30 μM and 90 μM of PMA007 (FIG. 9C) or 30 μM and 90 μM of PMA010 (FIG. 9D). The cells in FIGS. 9B, 9C, and 9D were grown in media without glucose, pyruvate, and glutamine. Scale bar=20 μm. FIG. 9E is a bar graph showing the quantification of the percent change in lysine succinylation (green) signal, from the immunofluorescence confocal micrographs of FIGS. 9B and 9C, in response to the treatment of PMA007. FIG. 9F is a bar graph showing the quantification of the percent change in lysine succinylation (green) signal, from the immunofluorescence confocal micrographs of FIGS. 9B and 9D, in response to the treatment of PMA010. Asterisks indicate statistical significance calculated at the raw data level (n=3); **P<0.01. ****P<0.0001 statistically compared to untreated. Statistical analysis used T-test.

FIG. 10 is a western blot showing the control Fb826 control normal levels and the reduction in specific signals of various ETC components in the propionic acidemia (PA) cell lines Fb859 and Fb900 and methylmalonic aciduria (MMA) cell line Fb857 compared to the control Fb826.

FIG. 11 are western blots showing correction in the signals of Complex III subunit Core 2 48-kDa (UQCRC2), Complex IV subunit II 22-kDa (COXII), and Complex I subunit NDUFB8 18-kDa that is most pronounced in the propionic acidemia (PA) cell line Fb859 compared to the methylmalonic aciduria (MMA) cell line Fb857.

FIG. 12A-12E are graphs showing effect of anaplerotic compounds treatment on mitochondrial energetics and radical oxygen species ROS detection. FIGS. 12A-12D are graphs showing changes in oxygen consumption rate (OCR) parameters following the addition of OXPHOS inhibitors to the media of PA patient cells, Fb859, treated with PMA007, PMA010, or individual components of D-Serine, D-Histidine, or adipic acid, at 30 μM and 90 μM for 72 hours. FIG. 12E shows ROS detected using MitoSOX Red as Superoxide indicator probe in cells grown in media that included the PMA007 or PMA010 or adipic acid, D-His, and D-Ser. Cell suspensions containing 1×10⁵ cells/mL were seeded in 96 well plates and treated with the compounds for 72 hours. Cells were washed and incubated for 15 min at 37° C., % 5% CO2 with 5 μM (MitoSOX Red; Invitrogen) for superoxide production measurement. After incubation, cells were washed, fresh media was added, and fluorescence was measured using microplate reader set to 520 nm excitation and 580 nm emission.

FIGS. 13A-13H are graphs showing oxygen consumption rate parameters changes in response to treatment of PA patient cells, Fb859, with varying concentrations of PMA010 (FIGS. 13A-13D) or PMA011 (FIGS. 13E-13H). Values are plotted as percentages to no treatment. Asterisks indicate statistical significance calculated at the raw data level (n=6-8); *P<0.1. **P<0.01. ***P<0.001. ****P<0.0001 statistically compared to untreated. Statistical analysis used T-test.

FIGS. 14A-14D are graphs showing the quantitation of propionyl-CoA alternative metabolites excreted in the media of FB859 patient and HEK293 PCCA^{-/-} cultured cells treated with PMA007 or PMA010 for 72 hours: propionylcarnitine (C3-carnitine; FIG. 14A), propionylglycine (C3-glycine; FIG. 14B), hydroxypropionylcarnitine (C3OH-carnitine; FIG. 14C), and hydroxypropionyl glycine (C3OH-glycine, FIG. 14D). Data are presented as percentage to untreated cells. In media of Fb859 cells, the control (Cntl, 0 μM) treatment with glucose and pyruvate, media propionylcarnitine was 0.17 μM, while in the untreated control, which had no glucose or pyruvate, and stripped FBS (no fat) included in the media, propionylcarnitine was 0.433 μM.

FIGS. 15A-15D are graphs showing the measurement of mitochondrial reactive oxygen species (ROS) production using MitoSOX Red as Superoxide indicator probe. FIG. 15A is a graph showing control fibroblast cell line FB826 treated with PMA010, PMA011, PMA019, and PMA020. FIG. 15B is a graph showing propionic acidemia (PA) patients' fibroblast cell linc Fb859 treated with PMA010, PMA011, PMA019, and PMA020. FIG. 15C is a graph showing propionic acidemia (PA) patients' fibroblast cells line Fb900 treated with PMA010, PMA011, PMA019, and PMA020. FIG. 15D is a graph comparing ROS production levels in response to treatment with PMA011 in Control fibroblast cell line (FB826), PA fibroblast cell line (Fb900), and glutaric acidemia II (GA II) patient fibroblast cell lines (Fb930, Fb961). Data are the average of readings from 3 replicate wells.

FIG. 16 are immunocytochemistry photomicrographs showing lysine hyposuccinylation in fatty acid oxidation disorders cell lines as observed in deficient cells (as designated) using anti-succinyllysine, anti-MTCO1 antibodies and DAPI for nuclei. Instrument: Zeiss LSM 710 confocal Microscopy. Magnification: 40×.

FIGS. 17A and 17B are tables showing the functional contribution of potential moieties that are anaplerotic in this class of compounds and mode of action and catabolic pathway.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. As used herein “a” and “an” refer to one or more.

As used herein, the term “comprising” is open-ended and may be synonymous with “including”, “containing”, or “characterized by”. The term “consisting essentially of” limits the scope of a claim to the specified materials or steps and those that do not materially affect basic and novel characteristic(s). The term “consisting of” excludes any element, step, or ingredient not specified in the claim. As used herein, embodiments “comprising” one or more stated elements or steps also include but are not limited to embodiments “consisting essentially of” and “consisting of” these stated elements or steps.

As used herein, the term “patient” or “subject” refers to members of the animal kingdom including but not limited to human beings and “mammal” refers to all mammals, including, but not limited to human beings.

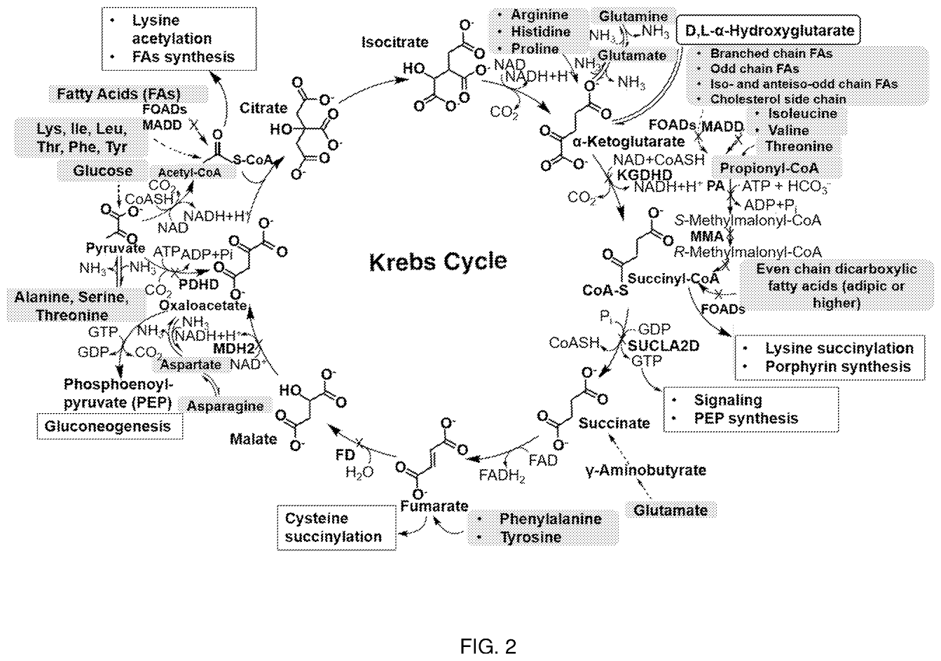

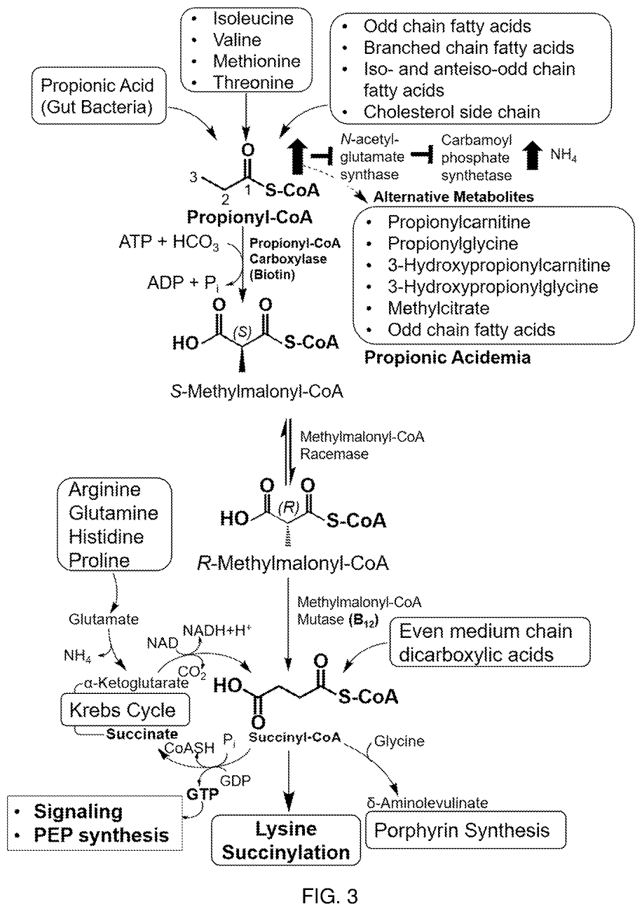

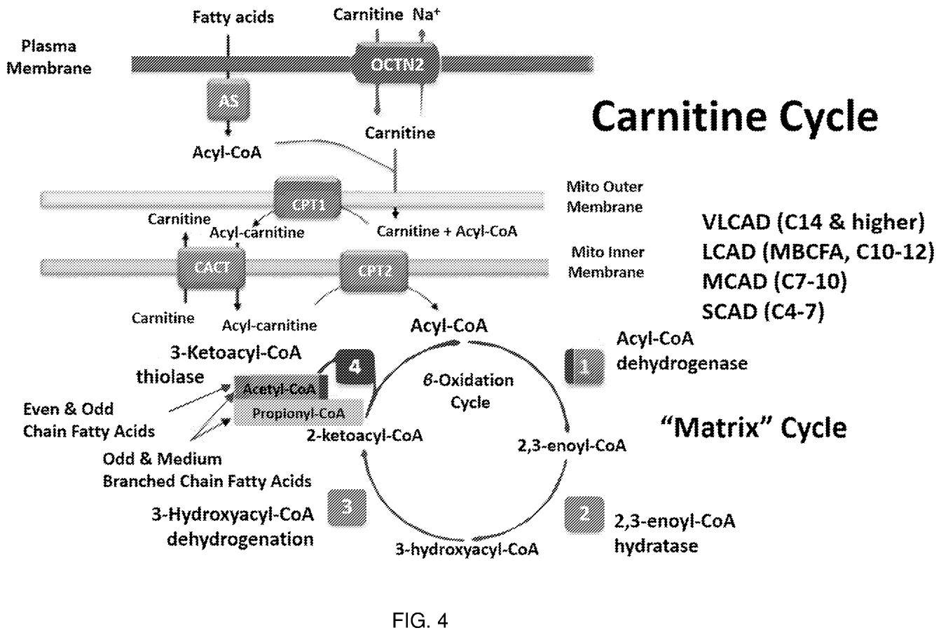

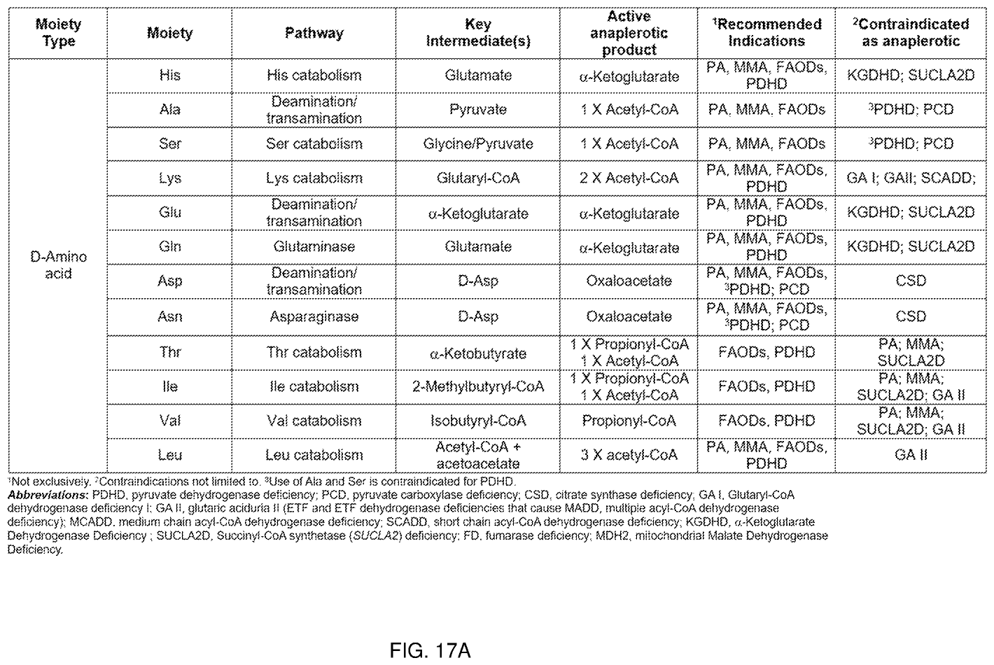

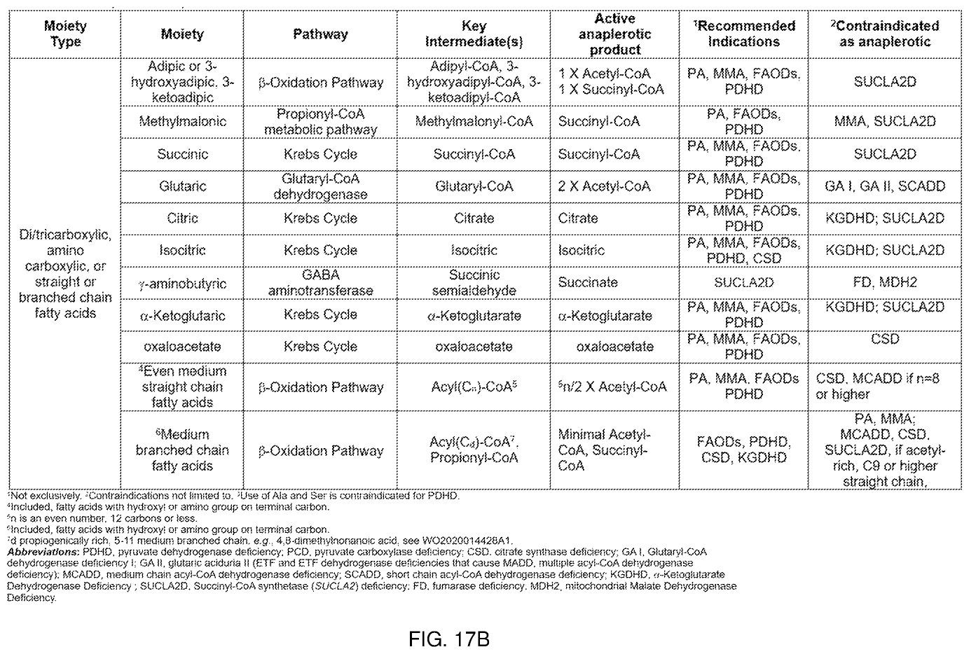

Provided herein are compounds and methods for treatment of certain metabolic diseases that include as a symptom or sequelae, or are caused by deficiencies in one or more anaplerotic reactions or pathways in a patient that produce an anaplerotic compound. Such diseases include, for example and without limitation, propionic acidemia, methymalonic acidemia, a fatty acid oxidation (e.g., β-oxidation) disorder, or specific Krebs cycle (FIG. 2) related enzyme deficiencies e.g., pyruvate dehydrogenase deficiency (see FIGS. 17A and 17B). The compounds useful in the methods described herein comprise an adipic acid or a methylmalonic acid moiety linked via amide bond to a single D amino acid, a D-dipeptide, or a D-tripeptide composed of the D amino acid form of His (histidine), Ala (alanine), Ser (serine), Lys (lysine), Glu (glutamate), Gln (glutamine), Asp (aspartate), Asn (asparagine), Leu (leucine), Isoleucine (Ile), Threonine (Thr), and Val (valine). Where Ser is present, it may be O-linked by its R group to form an ester, or its amine group to form a standard peptide bond. Further description of the various compounds are provided below. When administered to a patient, the compound is broken down into its constituents, which supplement anaplerotic reactions and pathways in a patient, and therefore address a cause, symptom, or sequelae of certain diseases. As different metabolic diseases result in different deficiencies in anaplerotic compounds, different compounds may be selected to support patients with different diseases.

It should be understood that metabolic pathways of different patients may break down compounds in a different manner depending, e.g., on patient genetics, nutrition, health, and activity, among other factors. That said, the compounds are expected to be metabolized primarily into intermediates that replenish certain anaplerotic compounds produced via anaplerotic pathways in a given patient. For example, adipic derivatives (that is, adipic acid or its derivatives, for example as described herein) are primarily expected to provide one acetyl-CoA and one succinyl-CoA in a patient. Methylmalonic acid is expected to supplement succinyl-CoA in a patient. D-amino acids are included in the structure as they are not directly used in protein synthesis or structure but are metabolized to their deaminated form. D-His is expected to increase α-ketoglutarate levels and thus serve as a succinyl-CoA precursor in the TCA cycle. D-Ala is expected to undergo deamination to form pyruvate, which may be broken down by pyruvate dehydrogenase for use in the TCA cycle, carboxylated to oxaloacetate (see FIG. 2), or can be used in gluconeogenesis. D-Ser may be deaminated and/or can be broken down to form pyruvate or glycine, or which can go other pathways. While all amino acids may be deaminated to the keto form and then re-aminated to the L-form, loss to the L-form for protein and other anabolic pathways should not significantly affect the bulk catabolized further.

As used herein, the terms “treating”, or “treatment” can refer to a beneficial or specific result, such as improving one of more functions, or symptoms of a disease. The terms “treating” or “treatment” can also include, but are not limited to, alleviation or amelioration of one or more symptoms of, or normalization of a clinically-relevant marker of propionic acidemia, methymalonic acidemia, or a fatty acid oxidation (e.g., β-oxidation) disorder. “Treatment” can also mean prolonging survival as compared to expected survival in the absence of treatment. As used herein, “percutaneous” refers to “through the skin.”

“Lower,” in the context of a disease marker or symptom, can refer to a clinically-relevant and/or a statistically significant decrease in such level. The decrease can be, for example, at least 10%, at least 20%, at least 30%, at least 40%, or more, down to a level accepted as within the range of normal for an individual without such disorder, or to below the level of detection of the assay. The decrease can be down to a level accepted as within the range of normal for an individual without such disorder, which can also be referred to as a normalization of a level. The reduction can be the normalization of the level of a sign or symptom of a disease, that is, a reduction in the difference between the subject level of a sign of the disease and the normal level of the sign for the disease (e.g., to the upper level of normal when the value for the subject must be decreased to reach a normal value, and to the lower level of normal when the value for the subject must be increased to reach a normal level). The methods described herein may include a clinically relevant reduction of any symptom or normalization of a clinical marker level for a disease, e.g. as described herein.

“Therapeutically effective amount,” as used herein, can include the amount of a compound as described herein that, when administered to a subject having a disease, can be sufficient to effect treatment of the disease (e.g., by diminishing, ameliorating or maintaining the existing disease or one or more symptoms of disease, including normalization of any relevant clinical marker). The “therapeutically effective amount” may vary depending on how the composition is administered, the disease and its severity and the history, age, weight, family history, genetic makeup, the types of preceding or concomitant treatments, if any, and other individual characteristics of the subject to be treated.

A “therapeutically-effective amount” can also include an amount of an agent that produces a local or systemic effect at a reasonable benefit/risk ratio applicable to any treatment. Compounds described herein may be administered in a sufficient amount to produce a reasonable benefit/risk ratio applicable to such treatment.

The phrase “pharmaceutically-acceptable carrier” as used herein can refer to a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier can be “acceptable” in the sense of being compatible with the other ingredients of the formulation and not injurious to the subject being treated. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (24) other non-toxic compatible substances employed in pharmaceutical formulations.

Pharmaceutically acceptable salts of any of the compounds described herein also may be used in the methods described herein. Pharmaceutically acceptable salt forms of the compounds described herein may be prepared by conventional methods known in the pharmaceutical arts, and include as a class veterinarily acceptable salts. For example, and without limitation, where a compound comprises a carboxylic acid group, a suitable salt thereof may be formed by reacting the compound with an appropriate base to provide the corresponding base addition salt. Non-limiting examples include: alkali metal hydroxides, such as potassium hydroxide, sodium hydroxide and lithium hydroxide; alkaline carth metal hydroxides, such as barium hydroxide and calcium hydroxide; alkali metal alkoxides, such as potassium ethanolate and sodium propanolate; and various organic bases such as piperidine, diethanolamine, and N-methylglutamine.

Non-limiting examples of pharmaceutically-acceptable base salts include: aluminum, ammonium, calcium, copper, ferric, ferrous, lithium, magnesium, manganic, manganous, potassium, sodium, and zinc salts. Salts derived from pharmaceutically acceptable organic non-toxic bases include, without limitation: salts of primary, secondary, and tertiary amines, substituted amines including naturally occurring substituted amines, cyclic amines, and basic ion exchange resins, such as arginine, betaine, caffeine, chloroprocaine, choline, N,N′-dibenzylethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine, and tris-(hydroxymethyl)-methylamine (tromethamine).

Non-limiting examples of pharmaceutically-acceptable acid salts include: acetate, adipate, alginate, arginate, aspartate, benzoate, besylate (benzenesulfonate), bisulfate, bisulfite, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, galacterate, galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate, and phthalate.

Multiple salts forms are also considered to be pharmaceutically-acceptable salts. Common, non-limiting examples of multiple salt forms include: bitartrate, diacetate, difumarate, dimeglumine, diphosphate, disodium, and trihydrochloride.

As such, “pharmaceutically acceptable salt” as used herein is intended to mean an active ingredient (drug) comprising a salt form of any compound as described herein. The salt form may confer improved and/or desirable pharmacokinetic/pharmodynamic properties of the compounds described herein.

Organic compounds can form complexes with solvents in which they are reacted or from which they are precipitated or crystallized. These complexes are known as “solvates.” A complex with water is known as a “hydrate.” Solvates of the compounds disclosed herein (e.g., compounds A-B, or B-A′) are contemplated. It will also be appreciated by those skilled in organic chemistry that many organic compounds can exist in more than one crystalline form. For example, crystalline form may vary from solvate to solvate. Thus, all crystalline forms of the compounds described herein (e.g., compounds A-B, or B-A′) or the pharmaceutically acceptable solvates thereof are within the scope of the present invention (See, generally, A. M. Healy, et al., Pharmaceutical solvates, hydrates and amorphous forms: A special emphasis on cocrystals, Adv. Drug Deliv. Rev. 2017 Aug. 1;117:25-46).

A “group” or “functional group” is a portion of a larger molecule comprising or consisting of a grouping of atoms and/or bonds that confer a chemical or physical quality to a molecule. A “residue” is the portion of a compound or monomer, such as, in the context of the present disclosure, a portion of an adipic acid, a 3-hydroxy-adipic acid, a 3-ketoadipic acid, a glutaric acid, a succinic acid, methylmalonic acid, a citric acid, an isocitric acid, α-ketoglutaric acid, an oxaloacetic acid, a heptanoic acid, a 2,6-dimethylheptanoic acid, a 4,8-dimethylnonanoic acid, a 6-amino-2,4-dimethylheptanoic acid, a 6-hydroxy-hexanoic acid, a 6-amino-hexanoic acid, D-Ser, D-Ala, D-Lys, D-Leu, D-His, D-Glu, D-Gln, D-Asp, D-Asn, D-Leu, D-Ile, D-Val, or D-Thr that remains in a larger molecule, such as a polymer chain or macromolecule described herein, after incorporation of that compound or monomer into the larger molecule. A compound that comprises a residue of another smaller compound, such as adipic acid, methylmalonic acid, D-Ser, D-Ala, D-Leu, D-Lys, and D-His is said to comprise that compound. A “moiety” is a portion of a molecule, and can comprise one or more functional groups, and in the case of an “active moiety” can be a characteristic portion of a molecule or compound that imparts activity, such as pharmacological or physiological activity, to a molecule as contrasted to inactive portions of a molecule such as esters of active moieties, or salts of active agents.

Provided herein are compounds useful in treating propionic acidemia, methylmalonic aciduria, and fatty acid oxidation disorders. The compounds may be referred to as anaplerotic compounds or anaplerotic agents for their utility in anaplerotic therapies, e.g., to replenish or supplement metabolic intermediates that may be diminished in a patient having a metabolic disease, such as a propionic acidemia, a methylmalonic aciduria, or a fatty acid oxidation disorders. The metabolic intermediate may be succinyl-CoA, or other intermediates. For example, the demand on α-ketoglutarate and succinyl-CoA for various biochemical functions makes it desirable to replenish the latter from other sources including amino acid metabolism and branched and odd chain fatty acids. The compounds have the structure A-B or B-A′, in which “A” is a dicarboxylate, a tricarboxylic, or a straight or branched-chain fatty acid selected from: an adipic acid, a 3-hydroxy-adipic acid, a 3-ketoadipic acid, a glutaric acid, a succinic acid, methylmalonic acid, a citric acid, an isocitric acid, α-ketoglutaric acid, an oxaloacetic acid, a heptanoic acid, a 2,6-dimethylheptanoic acid, a 4,8-dimethylnonanoic acid, a 6-amino-2,4-dimethylheptanoic acid, a straight chain fatty acid, or a branched-chain fatty acid and “A” is a straight or branched-chain fatty acid moiety selected from: a 6-hydroxy-hexanoic acid, a 6-amino-hexanoic acid, or a 6-amino-2,4-dimethylheptanoic acid.

“An adipic acid” is a substituted or unsubstituted straight-chain aliphatic dicarboxylic acid having six carbon atoms total. For example, the adipic acid may be substituted with a hydroxyl on the third carbon atom to form 3-hydroxyadipic acid or may be substituted with a keto on the third carbon atom to form 3-ketoadipic acid. “A glutaric acid” is a substituted or unsubstituted straight-chain aliphatic dicarboxylic acid having five carbon atoms total. For example, the glutaric acid may be substituted with a hydroxyl on the second carbon atom to form α-hydroxyglutaric acid or may be substituted with a keto on the second carbon atom to form α-ketoglutaric acid. “A succinic acid” is a substituted or unsubstituted straight-chain aliphatic dicarboxylic acid having four carbon atoms total. For example, the succinic acid may be substituted with a hydroxyl on the second carbon atom to form 2-hydroxysuccinic acid or may be substituted with a keto on the second carbon atom to form 2-ketosuccinic acid. “A citric acid” is a substituted or unsubstituted tricarboxylic acid bearing a hydroxy substituent on the second carbon. For example, the citric acid may be further substituted with a hydroxyl group to form hydroxycitric acid.

In one aspect or embodiment, the compound has the structure A-B and “A” comprises either an adipic acid moiety or a methylmalonic acid moiety in combination with “B”, which is an amino acid, a D-dipeptide, or a D-tripeptide of amino acids, comprising at least one an amino acid selected from D-Ser, D-Ala, D-His, D-Lys, D-Glu (D-glutamic acid), D-Gln (D-glutamine), D-Asp (D-aspartic acid), D-Asn (D-asparagine), D-Leu (D-leucine), D-Ile (D-isoleucine), D-Thr (D-threonine) or D-Val (D-valine). “A D-amino acid” is an amino acid wherein the stereogenic carbon alpha to the amino group has a D-configuration. “A D-dipeptide” is a peptide composed of two D-amino acid residues. “A D-tripeptide” is a peptide composed of three D-amino acid residues. In one aspect or embodiment, “B” is or comprises from 1-3 amino acids selected from D-Ser, D-Ala, D-Lys, D-Leu, and/or D-His. Alternatively, “B” is or comprises-D-Ser, -D-Ala,-D-His, -D-His-D-Ala-D-Ser, -D-His-D-Ser-D-Ala, -D-Ser-D-His-D-Ala, -D-Ser-D-Ala-D-His, -D-Ala-D-Ser-D-His, -D-Ala-D-His-D-Ser, -D-Ala-D-His-D-Ser, -D-His-D-Ala, -D-His-D-Ser, -D-Ser-D-His, -D-Ala-D-Ser, or -D-Ala-D-His, wherein D-Ser may be linked either by an amide bond (—N-linked) or an ester bond (—O-linked). Other alternatives are D-Leu, and/or D-Lys that may replace D-Ala or D-Ser.

In the compound structure A-B, A and B may be linked by an amide bond formed by reacting a carboxylic acid of A and the amine of B. In the case of D-Ser, A and B may be linked either by an amide bond or an ester bond, where the amide bond is formed by reacting a carboxylic acid of A and the amine of D-Ser and the ester bond is formed by reacting a carboxylic acid of A and the hydroxyl of D-Ser. In the compound structure B-A′, B and A′ may be linked either by an ester bond or an amide bond, where the amide bond is formed by reacting the carboxylic acid of B and the amine of A′ and the ester bond is formed by reacting the carboxylic acid of B and the hydroxyl of A′. D-amino acids are not metabolized as their L-counterparts, and, as such, the D-amino acids are subject to conversion to anaplerotic compounds that may be depleted in patients with metabolic diseases, such as propionic acidemia, methylmalonic acidurias, or fatty acid oxidation disorders.

Certain exemplary useful fatty acids for A and A′ include straight or branched, saturated or unsaturated C₄₋₂₄ fatty acids. Exemplary fatty acids are described in International Patent Publication No. WO 2020/014428 A1, the disclosure of which is incorporated herein by reference, including heptanoyl, 2-methylheptanoyl, 2,6-dimethylheptanoyl, 4,8-dimethylnonanoyl, 6-amino-2,4-dimethylheptanoyl, linolenoyl, docosahexaenoyl, or eicosapentaenoyl fatty acid moieties. Other useful fatty acid moieties include a C_{18:3}, C_{20:5}, or C_{22:6} fatty acid moiety having double bonds at positions ω3 and ω6 of the C_{18:3}, C_{20:5}, or C_{22:6} fatty acid moiety. The fatty acids may be ω3 or ω6 (e.g., n−3) fatty acids, α-linolenoyl (e.g., C_{18:3}), docosahexaenoyl (e.g., C_{22:6}), and eicosapentaenoyl (e.g., C_{20:5}) fatty acids. The fatty acids may be odd-numbered, such as heptanoyl or nonanoyl, or even numbered, such as linolenoyl, docosahexaenoyl, or eicosapentaenoyl. These long chain unsaturated fatty acids may be useful for specific fatty acid oxidation disorders, e.g., medium chain acyl-CoA dehydrogenase deficiency (MCADD). The fatty acids may be a medium chain (e.g., C₆₋₁₂, but not for MCADD), or short chain (e.g., C₁₋₅) fatty acid.

Serine (Ser), other than the carboxyl, includes two reactive groups that may be used to link the serine to a larger molecule, namely an amine group at the Cα position (a in the following structures) or a carboxyl group (b in the following structures). Ser may be conjugated (covalently linked) to a carboxyl group of the adipic acid moiety or the methylmalonic acid moiety via its amine, forming an amide linkage (“a” in the following structures), or via the hydroxyl group of its R-group, forming an ester linkage (“b” in the following structures), and in which case, the O-linked (ester-linked) D-Ser is referred to herein as —O-D-Ser. Note in the following, the terminal amino acid is the D-Ser, but that one or more additional amino acids optionally may be linked to the Ser via the carboxyl group to form an amide (peptide) bond. The dashed bond line refers to a bond linking the depicted moiety to the remainder of the molecule (not shown). Chirality is not depicted.

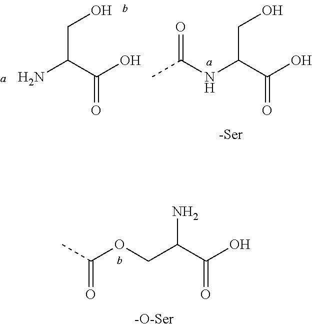

The compound may have the structure:

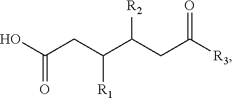

for example adipic-R₃ (as shown, an adipic acid moiety linked to R₃), where R₁ and R₂ are, independently H, OH (hydroxyl), or O (e.g., forming a carbonyl or keto moiety, with the O connected via a double bond), where at least one of R₁ and R₂ is H, and R₃ is linked to the adipic acid moiety via an ester (—O-linked) or amide (—N-linked) bond, and can comprise any combination of from one to three amino acids selected from: D-Ser, D-Ala, D-His, D-Lys, D-Glu, D-Asp, D-Asn, D-Leu, or D-Val. For example, and without limitation, considering inclusion of one or more of D-Ser, D-Ala, and D-His, R₃ may be: -D-Ser, —O-D-Ser, -D-Ala, -D-His, -D-His-D-Ala-D-Ser, -D-His-D-Ala-O-D-Ser, -D-His-D-Ser-D-Ala, -D-His-O-D-Ser-D-Ala, -D-Ser-D-His-D-Ala, —O-D-Ser-D-His-D-Ala, -D-Ser-D-Ala-D-His, —O-D-Ser-D-Ala-D-His, -D-Ala-D-Ser-D-His, -D-Ala-O-D-Ser-D-His, -D-Ala-D-His-D-Ser, -D-Ala-D-His-D-Ser, -D-His-D-Ala, -D-His-D-Ser, -D-His-O-D-Ser, -D-Ser-D-His, -D-Ala-O-D-Ser, or -D-Ala-D-His. Likewise, all permutations of from 1-3 amino acids selected from: D-Ser, D-Ala, D-His, D-Lys, D-Glu, D-Gln, D-Asp, D-Asn, D-Leu, and D-Val are contemplated for R₃. Further in each permutation, the adipic acid group may be substituted with another dicarboxylate or a straight or branched-chain fatty acid selected from: a different adipic acid, methylmalonic acid, succinate, glutaric acid, α-ketoglutarate, an even chain fatty acid, or a branched-chain fatty acid.

For example, where the serine residue is D-Ser is linked to the adipic moiety residue via its R-group (—CH₂OH, referred to herein as —O-D-Ser-), the compound has, e.g., the structure:

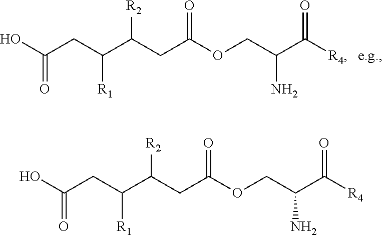

e.g., adipic-O-Ser-R₄ (left) or adipic-O-D-Ser-R₄ (right), where R₄ is or comprises: —OH, -His-Ala, -Ala-His, -Ala, or-His, where one, two, or all of the present Ser, Ala or His amino acid residues is in the D configuration, for example R₄ is or comprises: —OH, -D-His-D-Ala, -D-Ala-D-His, -D-Ala, or -D-His.

The compound may have the structure:

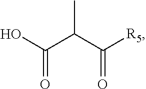

for example methylmalonic-R₅, where R₅ is linked to the methylmalonic acid moiety via an ester (—O-linked) or amide (—N-linked) bond, and can comprise can comprise any combination of from one to three amino acids selected from: D-Ser, D-Ala, D-His, D-Lys, D-Glu, D-Asp, D-Asn, D-Leu, or D-Val. For example, and without limitation, considering inclusion of one or more of D-Ser, D-Ala, D-Lys, D-Leu, and D-His, R3 may be: : -D-Ser, —O-D-Ser, -D-Ala, -D-His, -D-His-D-Ala-D-Ser, -D-His-D-Ala-O-D-Ser, -D-His-D-Ser-D-Ala, -D-His-O-D-Ser-D-Ala, -D-Ser-D-His-D-Ala, -O-D-Ser-D-His-D-Ala, -D-Ser-D-Ala-D-His, —O-D-Ser-D-Ala-D-His, -D-Ala-D-Ser-D-His, -D-Ala-O-D-Ser-D-His, -D-Ala-D-His-D-Ser, -D-Ala-D-His-D-Ser, -D-His-D-Ala, -D-His-D-Ser, -D-His-O-D-Ser, -D-Ser-D-His,-D-Ala-O-D-Ser, -D-Ala-D-His, D-Leu-D-His, or -D-Lys-D-His. Likewise, all permutations of from 1-3 amino acids selected from: D-Ser, —O-D-Ser, D-Ala, D-His, D-Lys, D-Glu, D-Gln, D-Asp, D-Asn, and D-Leu are contemplated for R₅.

For example, where the serine residue is D-Ser is linked to the methylmalonyl residue via its R-group (—CH₂OH, referred to herein as —O-D-Ser-), the compound has, e.g., the structure:

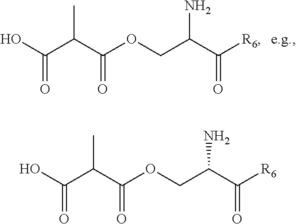

e.g., methylmalonic-O-Ser-R₆, (left) or methylmalonic-O-D-Ser-R₆ (right), where R₆ is or comprises: —OH, -His-Ala, -Ala-His, -Ala, or -His, where one, two, or all of the present Ser, Ala, or His amino acid residues is in the D configuration, for example R₆ is or comprises: —OH, -D-His-D-Ala, -D-Ala-D-His, -D-Ala, or -D-His.

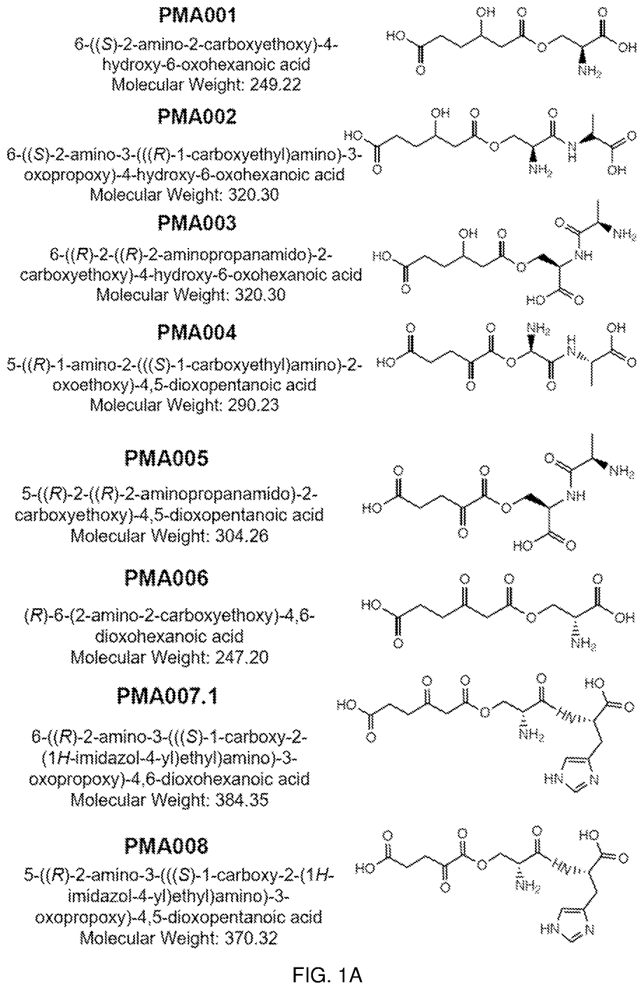

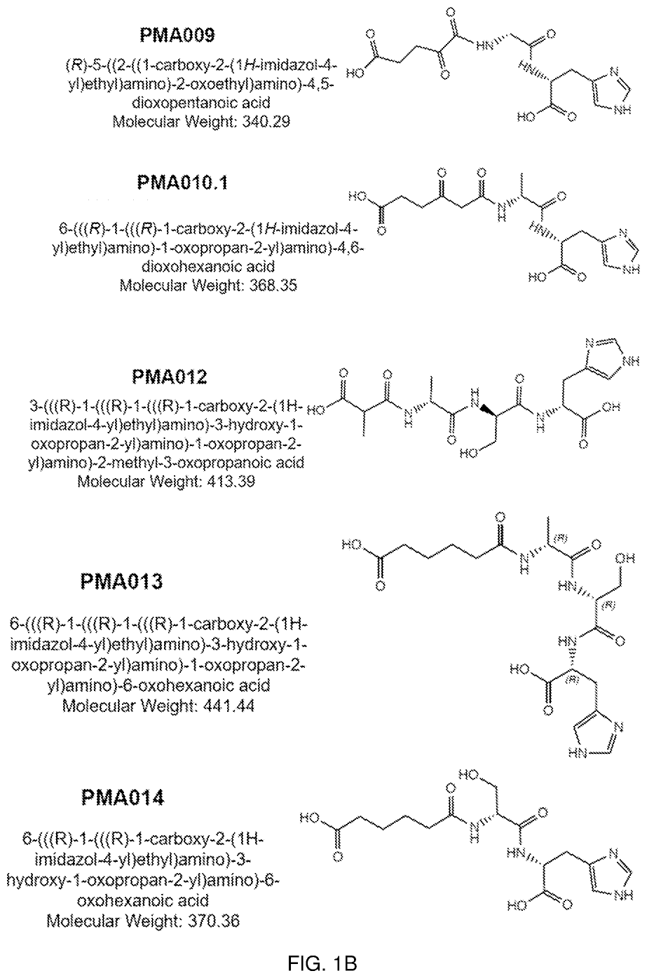

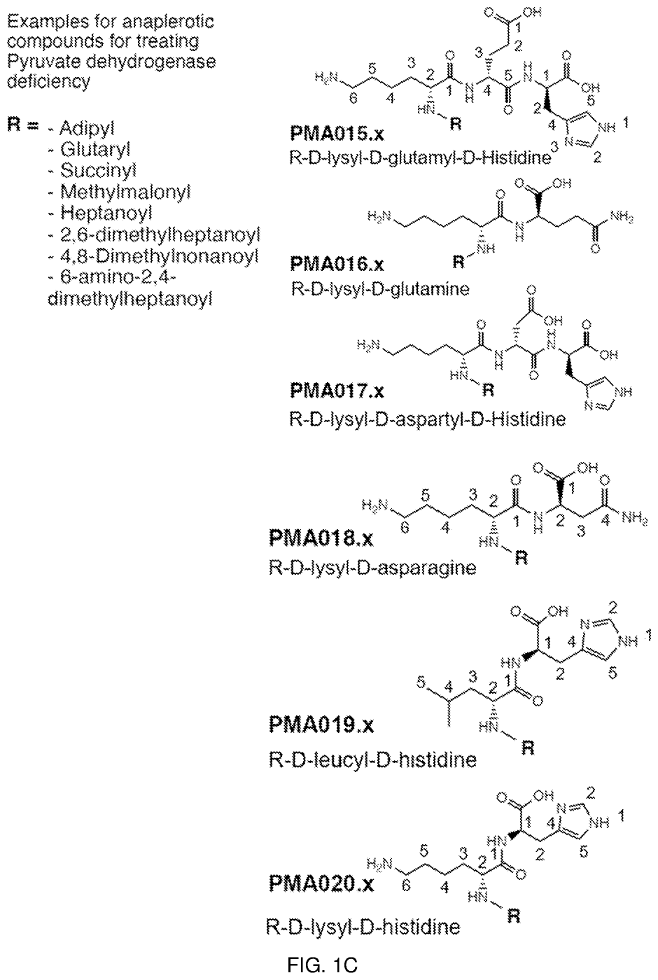

Non-limiting examples of such compounds include: PMA001, e.g., 4-hydroxyladipic acid-O-D-Ser; PMA002, e.g., 4-hydroxyladipic acid-O-D-Ser-D-Ala; PMA003, e.g., 4-hydroxyladipic acid-O-D-Ser-D-Ala; PMA004, e.g., 2-ketoglutaric acid-D-2-hydroxyglycine-D-Ala; PMA005, e.g., 2-ketoglutaric acid-O-D-Ser-D-Ala; PMA006, e.g., 4-ketoadipic acid-O-D-Ser; PMA007.1, e.g., 4-ketoadipic acid-O-D-Ser-D-His; PMA008, e.g., 2-ketoglutaric acid-O-D-Ser-D-His; PMA009, e.g., 2-ketoglutaric acid-D-Ala-D-His; PMA010.1, e.g., 4-ketoadipic acid-D-Ala-D-His; PMA007, e.g., methylmalonic acid-O-D-Ser-D-His; PMA011, e.g., methylmalonic acid-D-Ala-D-His; PMA012, e.g., methylmalonic acid-D-Ala-D-Ser-D-His; PMA013, e.g., adipic acid-D-Ala-D-Ser-D-His; PMA014, e.g., adipic acid-D-Ser-D-His, and PMA015, PMA016, PMA017, PMA018, PMA019, or PMA020 which include an R group selected from adipyl, glutaryl, succinyl, heptanoyl, 2,6-dimethylheptanoyl, 4,8-dimethylnonanoyl, and 6-amino-2,4-dimethylheptanyl, linked to: R-D-lysyl-D-glutamyl-D-histidine (PMA015), R-D-lysyl-D-glutamine (PMA016), R-D-lysyl-D-aspartyl-D-histidine (PMA017), or R-D-lysyl-D-asparagine (PMA018), R-D-leucyl-D-histidine (PMA019), and R-D-lysyl-D-histidine (PMA020). FIGS. 1A-1C, 5, and 6 depict these exemplary structures.

FIGS. 17A and 17B provide further examples of useful moicties for A and B, and describes conditions the particular moiety expected to be useful in treating various indications, and includes contraindications. In further detail, FIGS. 17A and 17B describe the functional contribution of potential moieties that are anaplerotic in this class of compounds and mode of action and catabolic pathway. Potential D-amino acids can be used in conjunction with specific fatty acids. For PA and MMA, propiogenic amino acids and propiogenic fatty acids, including branched and odd-chained amino acids and fatty acids, are contraindicated.

In treating propionic acidemia or methylmalonic acidura, the method may further comprises co-administering to the patient, along with the compound described above, an inhibitor that blocks the formation of the propionyl-CoA acid or methylmalonyl-CoA, in propionic acidemia or methylmalonic aciduria, respectively. Co-administration refers to administration of a second active agent during the course of therapy with a first active agent, e.g., as described above. The second active agent may be administered before, after, or concurrently with the first active agent, and may be formulated in the same unit dosage form. In one example, the inhibitor that blocks the formation of the propionyl-CoA acid or methylmalonyl-CoA is 2,2-dimethybutyric acid, sodium 2,2-dimethylbutanoate, or 2,2-dimethylbutanoate formulation, or any inhibitor that restrict the function of propionyl-CoA carboxylase.

Also, as described herein, propionyl-CoA/methylmalonyl-CoA (P-CoA/MM-CoA) formation inhibitors may be administered to the patient as an adjuvant therapy, along with the compounds described herein. For example, the P-CoA/MM-CoA formation inhibitor may be HST5040 (see, e.g., Armstrong et al. “Identification of 2,2-Dimethylbutanoic Acid (HST5040), a Clinical Development Candidate for the Treatment of Propionic Acidemia and Methylmalonic Acidemia”, J. Med. Chem., 2021, 64:5037-5048).

Propionic acidemia: Propionic acidemia is an autosomal recessive metabolic disorder. It is caused by deficiency of propionyl-CoA carboxylase (PCC), a mitochondrial enzyme that catalyzes conversion of propionyl-CoA to S-methylmalonyl-CoA. Propionyl-CoA is end-product of the catabolismof valine, isoleucine, methionine, and threonine and odd-chain fatty acids β-oxidation and is also a side-product of the B-oxidation of branched-chain fatty acids including phytanic and pristanic acids. PCC deficiency cause accumulation of propionyl-CoA, which thiolyses releasing toxic amounts of propionic acid, and concomitant deficiency of its downstream pathway's end-product, succinyl-CoA, an intermediate of the TCA cycle and critically causing cycle dysfunction. Not surprisingly, some fatty acid β-oxidation disorders and propionic acidemia present with cardiomyopathy as a common phenotype.

PA clinical features: The disorder may present in the first week of life with feeding difficulties, lethargy, vomiting, and life-threatening acidosis, hypoglycemia, hyperammonemia, and/or bone marrow suppression with high mortality in early onset. Serious hyperammonemia contributes to the encephalopathy of acute illness of newborns. The hyperammonemia is attributed to N-acetylglutamate synthesis inhibition by excess propionyl-CoA, the activator of carbamoyl phosphate synthetase. Mortality is high in early disease onset. Equally common is a more chronic course, which presents in the first months of life with poor feeding and episodes of vomiting, infection-induced ketoacidosis, failure to thrive, and osteoporosis, severe enough to cause pathologic fractures. Developmental retardation attributed to hyperammonemia or chronic illness, is common. Metabolic strokes due to acute degeneration of the basal ganglia may occur during or between episodes of ketoacidosis. Cardiomyopathy, which may be rapidly fatal, occurs frequently and does not respond to carnitine. Pancreatitis is a recognized complication of the disease as well as chronic kidney disease.

Treatment is directed to treating shock, acidosis, hypoglycemia, and hyperammonemia with fluids, bicarbonate, glucose, and dialysis. Restriction of dietary natural protein (or of propiogenic amino acids) to amounts necessary to support normal growth and development is indicated, and usually results in natural protein intake less than 1 g/kg/day. Liver transplant is commonly resorted to and decreases the risk for episodes of metabolic decompensation and can reverse cardiomyopathy.

Anaplerotic therapy: Proof of concept that anaplerotic compounds can be therapeutic was reported more than two decades ago using L-malate as an intermediate of the Krebs cycle. The study was carried out in vitro using rat liver mitochondrial assay. The researchers reported that the L-malate treatment has reduced the propionyl-CoA levels in the mitochondrial matrix by 62% and propionate level by 46%. In a clinical study initiated in 2008 anaplerotic compounds such as glutamine, citrate, and ornithine α-ketoglutarate were tested on PA patients and the authors concluded that citrate supplementation can be beneficial in some cases as it helps replenish depleted Krebs cycle intermediates. Despite the positives in these studies, the mode of delivery of these anaplerotic compounds to their target site in mitochondria has likely been the major impediment for these compounds to bring effective therapeutic outcome.

Methylmalonic acidurias (MMA). Methylmalonic aciduria can be caused by an inherited deficiency of methylmalonyl-CoA mutase, the downstream enzyme to PCC, and is an adenosylcobalamin-requiring enzyme. It converts L-methylmalonyl-CoA to succinyl-CoA, or in the metabolic pathway that catalyzes the biosynthesis of adenosylcobalamin from vitamin B12. When the latter defect occurs in a proximal step that also impairs the synthesis of methylcobalamin, homocysteine accumulates behind a block in N₅-methyltetrahydrofolate: homocysteine methyltransferase.

MMA clinical presentation: Complete methylmalonyl-coA mutase deficiency clinical presentation includes symptoms common with propionic acidemia. Severe ketoacidosis, hyperammonemia, and thrombocytopenia in the first days or weeks of life are common. Patients with some residual mutase activity manifest later with a variety of symptoms including intermittent ataxia, recurrent vomiting, failure to thrive, and developmental delay. Whether severe or mild, life threatening episodes of decompensation may occur typically due to minor intercurrent illnesses.

Patients with cblA and cblB defects usually have isolated methylmalonic aciduria but somewhat milder disease. Mutations in the SUCLA gene, which encodes an ATP forming subunit of the Krebs cycle enzyme succinyl-CoA ligase, is a novel cause of methylmalonic aciduria. Affected patients have a severe phenotype including hypotonia, muscle atrophy, hyperkinesia, mental retardation, growth failure, central and cortical atrophy of the brain, and basal ganglia atrophy. Mutations in the CblC gene cause combined methylmalonic aciduria and homocystinemia. CbIC deficiency most commonly presents in infancy with severe clinical manifestations including basal ganglia necrosis, microcephaly, failure to thrive, intellectual disability, retinopathy, and megaloblastic anemia. CblD mutations can cause combined disease as seen in CblC deficient patients, but variants with isolated methylmalonic aciduria as well as isolated homocystinemia have been identified. Patients with defects in the ChlE and CblG groups are deficient only in methylcobalamin biosynthesis, and have homocystinuria without methylmalonic aciduria. CblF deficiency results in defective transport of B12 out of lysosomes and a combined methylmalonic aciduria and homocystinemia. As in propionic acidemia, treatment in episodes of acute metabolic decompensation is directed first to treating shock, acidosis, hypoglycemia, and hyperammonemia, followed by restriction of protein (specifically, propiogenic amino acids). Carnitine is used to treat secondary carnitine deficiency. Some patients treated in this manner do well, but many do not and die in early childhood, often during an episode of ketoacidosis. Liver or liver/kidney transplantation reduces the severity but does not cure disease.

Defects in long-chain fatty acid oxidation cause significant decrease in lysine succinylation of mitochondrial proteins and is part of the pathomechanism. Collectively as a group, defects in MCAD, VLCAD, LCHAD, TFP, and CPT II are the most common metabolic defects identified through newborn screening using tandem mass spectrometry.

Therapies for treating mitochondrial fatty acid β-oxidation deficient patients, e.g., treatment of MCAD, VLCADD, LCHADD, TFPD, or CPT IID deficient patients are required to minimize less life-threatening episodes of decompensation that occur in most that require frequent hospitalization in the first 2-3 years of life. In young patients, adults, and older patients, a healthy exercise-tolerant lifestyle is also important to avoid other common late onset diseases. U.S. Pat. No. 8,399,515 describes C5 and C15 fatty acids useful in treatment of fatty acid disorders, and WO/2000045649 describes C7 fatty acids, e.g., n-heptanoic acid for treatment of fatty acid disorders which lead to the development of triheptanoin. Additional and/or superior compositions for treatment of fatty acid disorders are desirable. Medium branched chain fatty acids have also been reported as alternative treatment to compensate for the depletion of succinyl-CoA (US WO2020014428 A1).

### EXAMPLES

There are three major pathways that maintain succinyl-CoA at its physiologically optimum level. These are the TCA cycle, amino acid catabolismand branched and odd chain fatty acid β-oxidation. Proper function of these three pathways is critical. Deficiency in enzymes that catalyze any of the three pathways results in disease. The propiogenic amino acid (valine, isoleucine, methionine, and threonine) catabolismand branched and odd chain fatty acid β-oxidation generate propionyl-CoA. In the absence of proper supply of propionyl-CoA from either of these pathways, or, as in the case of PA and MMA, the supply of succinyl-CoA is severely affected, it is possible to replenish the succinyl-CoA through the TCA cycle, provided the administered compounds metabolic path will be committed to breakdown and are not directly useful as building blocks for biosynthesis of active larger molecules. The building blocks of the compounds proposed include D-histidine plus D-alanine or D-serine, where the former will directly supply α-ketoglutarate, the precursor of succinyl-CoA in the TCA cycle, and the D-alanine will commit to deamination to pyruvate, which may breakdown by pyruvate dehydrogenase to be utilized fully in the TCA cycle or still can be used in gluconeogenesis. D-Serine is to deaminate and may be re-aminated back to L-serine or breakdown to glycine that may be useful for conjugation with propionic acid or can go other pathways. Adipic acid or its derivatives (see below) are hypothesized to lead to provide one acetyl-CoA and one succinyl-CoA.

### Compounds

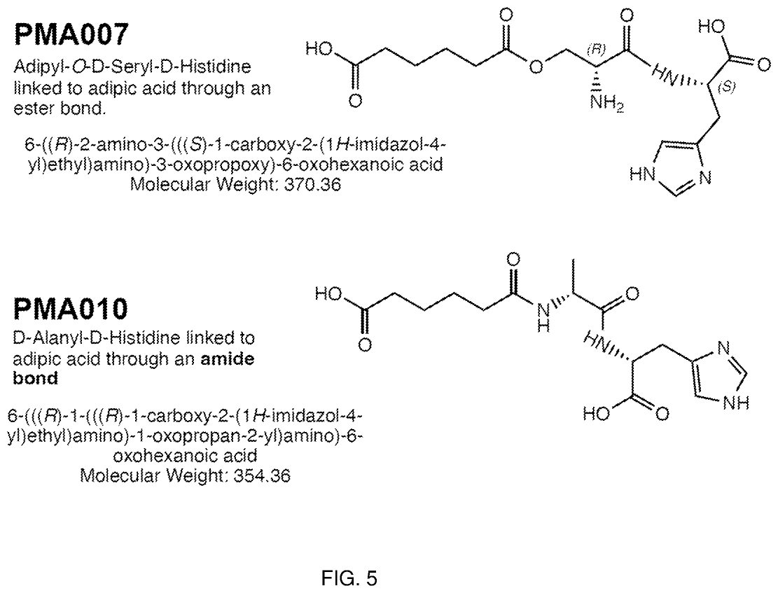

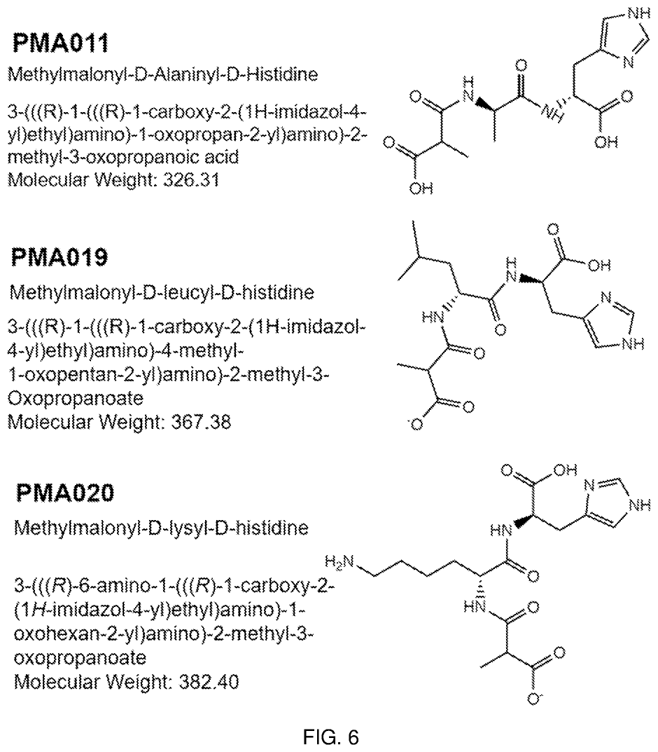

Pertaining to this disclosure three compounds, dicarboxylic acyl-D-dipeptides, designed as follows: (1) top structure, FIG. 5, adipic-D-O-Ser-D-His (PMA007), where the linkage between adipic acid and D-Ser is through an ester bond and the D-Ser and D-His are linked by a peptide bond. (2) The bottom structure, FIG. 5, adipic-D-Ala-D-His (PMA010), where the linkage between the three moieties is through a peptide bond. (3) The top structure, FIG. 6, methylmalonic-D-Ala-D-His (PMA011), where the linkage between the three moieties is through a peptide bond. In addition, limited testing was done and data is available for two compounds, PMA019, middle structure, FIG. 6, methylmalonic-D-Leu-D-His and PMA020 , bottom structure, FIG. 6, methylmalonic-D-Lys-D-His.

### MATERIALS AND METHODS

Compounds synthesis: The dicarboxylic acyl-D-dipeptides; PMA007, PMA010, PMA011, PMA019, and PMA020 were synthesized by solid-phase synthesis on a Liberty Microwave Synthesizer (CEM Corporation, 3100 Smith Farm Road, Mathews, NC 28106) using FMOC synthesis protocols. (Peptide & Peptoid Synthesis Facility, University of Pittsburgh Health Sciences Core Research Facilities). Briefly, syntheses were performed by stepwise addition of activated amino acid residues to the solid support (Wang resin) starting from the carboxy terminus to the amino terminus. Activation of amino acids were performed by DIC-Oxyma chemistry. At the end of the syntheses the peptides PMA010 was conjugated to adipic acid and methylmalonate, respectively, using PyBop/DIPEA chemistry, overnight with the coupling efficiency being confirmed by ninhydrin testing. Additionally, adipic acid was coupled to the Ser-hydroxyl group of the compound PMA007 using PyBop/DMAP coupling conditions. At the end peptides were cleaved off the resin by reagent B (95% TFA, 2.5% TIS and 2.5% H₂O) and subjected to multiple ether extractions. The crude peptides were analyzed, characterized, and purified by Reversed-Phase High Performance Liquid Chromatography (RP-HPLC, 486 and 600E by Waters Corporation) and later confirmed to have the correct mass on a Bruker Ultraflextreme MALDI Tof/Tof mass spectrometer.

Cell lines and culture: All the proof-of-concept testing was carried out in vitro on fibroblast cells from patient(s) with propionic acidemia and methymalonic acidemia to test these two compounds.

TABLE 1
Fibroblast cell lines designation, genotype, and
phenotype data used in this current study.

| | | | | |
|---|---|---|---|---|
| | Patient | | | |
| Cell | deficiency | Defective | | |
| lines | designation | protein | Genotype | Comments |

| | | | |
|---|---|---|---|
| FB859 | Propionic | Propionyl-CoA | |
| | acidemia | Carboxylase | |
| FB857 | MMA | Methylmalonyl-CoA | Unrespon- |
| (GM0050) | | Mutase | sive to |
| | | | B12 |
| FB858 | MMA | Methylmalonyl-CoA | |
| (GM01673) | | Mutase | |
| FB900 | Propionic | Propionyl-CoA | |
| | acidemia | Carboxylase | |
| FB859 | Propionic | Propionyl-CoA | |
| | acidemia | Carboxylase | |
| FB930 | Glutaric | Electron transfer | |
| | Aciduria II | flavoprotein | |
| FB961 | Glutaric | Electron transfer | |
| | Aciduria II | flavoprotein | |
| FB826 | N/A | N/A | control |

A control fibroblast cell line, FB826, was used to estimate the amount of detectable succinyllysine in “normal” cells using anti-succinyllysine antibody.

Immunohistochemistry: Patient cells were grown in glass coverslips at a seeding density of 3-5×10⁴ cells. The media for untreated and treatment groups had no glucose, glutamine or pyruvate, and lipid stripped FBS was included instead of regular FBS. NIH Image J software was used to quantify the fluorescence intensity from 50-60 cells per group. Immunocytochemistry of propionic acidemia patient (Fb859) fibroblast cells lines stained for anti-succinyllysine (Green), anti-MTCO1 (Red), or anti-TOMM20 antibodies, and DAPI (Blue) for nuclei. Instrument: Zeiss LSM 710 confocal Microscopy. Statistical analysis used Tukey multiple range test. ****P<0.0001, [PMA010] (90 μM) treatment group statistically compared to control cells grown in the presence of regular complete DMEM media, untreated and [PMA010] (30 μM) cells grown in the absence of glucose/pyruvate/L-glutamine.

Measurement of Mitochondrial Respiration Seahorse: Fb859 cells were grown in regular DMEM media until 85-90% confluency. The cells were harvested and seeded at a concentration of 25,000 cells/80 μl (n=8 replicates) in poly-D lysine coated XFe seahorse 96well plate in regular DMEM media and incubated overnight at 37° C. in a 5% CO₂ incubator. Following day, the culture media was replaced in designated wells with the following for 72 h:180 μl of Specialty Media A (seahorse XFDMEM base media+10% fat stripped FBS+Glucose+sodium pyruvate+L-glutamine+0.5 mM carnitine). 180 μl of Specialty Media B (scahorse XFDMEM base media+10% stripped FBS+0.5 mM carnitine and no added glucose, sodium pyruvate or glutamine). 180 μl of Specialty media B containing experimental compounds. After 72 h incubation the Specialty media A & B was replaced in respective designated wells with 180 μL Seahorse XF DMEM base media containing glucose, sodium pyruvate. L-glutamine or completely devoid of glucose, sodium pyruvate, L-glutamine and incubated at 37° C. in a non-CO₂ incubator for 1 h before measurement. OCR was determined (Seahorse XF MitoStress test). Finally, cells were lysed, and protein content was determined. Data is normalized to protein concentration and OCR is reported in pmol/min/mg protein.

Detection of ETC components by western blots: Same as above, media A for control, and media B for untreated and treated cells in T75 flasks. Amounts of compounds were added appropriately directly to cell culture media when the cultures were about 85-90 confluent. The cultures were allowed to grow for 72 h at 37° C./5% CO₂, and then harvested. Harvested cell pellets were stored at −80° C. until immune and enzymatic assay and western blot analyses of OXPHOS subunits. One to 1.5 ml media samples were also stored at −80° C. for.

Measurement of Mitochondrial Respiration: Control Fb826 and Patient Fb859 cells were grown in regular DMEM media without adding extra glutamine until 85-90% confluency. The cells were harvested and seeded at a concentration of 25,000 cells/80 μL (n=8 replicates) in poly-D lysine coated XFe Seahorse 96 well plate in regular DMEM media without adding extra glutamine and incubated overnight at 37° C. in a 5% CO₂ incubator. Following day, the culture media was replaced in designated wells with the following for 72 h: (1) 180 μL of Specialty media A (Seahorse XFDMEM base media with added 10% stripped (lipid free) FBS. glucose, sodium pyruvate, L-glutamine, and 0.5 mM carnitine). (2) 180 μL of specialty media B (scahorse XFDMEM base media, 10% stripped FBS, and 0.5 mM carnitine and devoid of glucose, sodium pyruvate and Glutamine). (3) 180 μL of Specialty media B containing “reference compounds” (30, 90, 150 μM) D-serine, D-histidine, or adipic acid and combination of 90 μM D-serine +D-histidine, and adipic acid, or the experimental compounds 30 μM or 90 μM PMA007 or PMA010. After 72 h incubation the Specialty media A & B was replaced in respective designated wells with 180 μL Seahorse XF DMEM base media containing glucose, sodium pyruvate, and L-glutamine, or devoid of these three ingredients or the experimental compounds and incubated at 37° C. in a non-CO₂ incubator for 1 h before measurements on the XFe Seahorse instrument from Agilent. Oxygen consumption rate (OCR) was determined (Seahorse XF MitoStress test). Finally, cells were lysed, and protein content was determined. Data was normalized to protein concentration and OCR is reported in pmol/min/mg protein.

Acylcarnitine levels determination: Media from control, Fb826, and treated and untreated patient, Fb859, cells were collected from either the immunohistochemistry cultures or the Seahorse oxygen consumption 96-well plate assays and analyzed for the levels of short chain acylcarnitines plus propionylglycine and hydroxypropionylglycine using a triple quadrupole API4000 mass spectrometer (AB Sciex™, Framingham, MA) equipped with a ExionLC™ 100 HPLC system (Shimadzu Scientific Instruments™, Columbia, MD) as outlined as previously published.

Measurement of mitochondrial ROS production using MitoSOX Red as superoxide indicator probe: control (Fb826), PA patient (Fb859, Fb900) and GA II patient (Fb930, Fb961) fibroblasts were grown in regular DMEM media containing glucose/pyruvate/L-glutamine until 85-90% confluency. The cells were harvested and seeded at a concentration of 25,000 cells/80 μl (n=3) in cell culture treated black wall 96 well microplate with clear bottom in regular DMEM media containing glucose/pyruvate/L-glutamine and incubated overnight at 37° C. in a 5% CO₂ incubator. Following day, the culture media was replaced in designated wells with the following for 72 h: 150 μl regular DMEM (Glucose+10% FBS+sodium pyruvate+L-glutamine+0.5 mM carnitine). 150 μl of Regular DMEM containing experimental compounds (180 and 640 μM) PMA010 or PMA011 or PMA 019 and (640 μM) PMA020 in control and propionic acidemia patient fibroblasts. 150 μl of regular DMEM containing experimental compounds (600 and 1200 μM) PMA011 in Fb930 and (1200 μM) PMA011 in Fb961 GA II patient fibroblast. After 72 h incubation the wells were washed twice with 150 μL of Seahorse XFDMEM base media and in respective designated wells the following Assay buffer (150 μL of seahorse XFDMEM base media containing glucose, sodium pyruvate and Glutamine) was used along with 5 μM MitoSOX reagent and incubated at 37° C. non-CO₂ incubator for 15 minutes protected from light. After 15 minutes incubation the cells were washed twice with 150 μL of seahorse XFDMEM base media and replaced with measurement buffer, which is same as the wash buffer. The fluorescence was monitored with excitation at 520 nm and emission at 580 nm. Finally, cells were lysed, and protein content was determined. Data is normalized to protein concentration and fluorescence is reported as MitoSOX Red (AFU)/mg protein.

### RESULTS AND DISCUSSION

Five biomarkers were evaluated to measure the effectiveness of the compounds on the cellular level in terms of lysine succinylation, specific OXPHOS subunits western blot pattern, O₂ consumption rate parameters, C3 acylcarnitine species analysis, and effect on reactive oxygen specics.

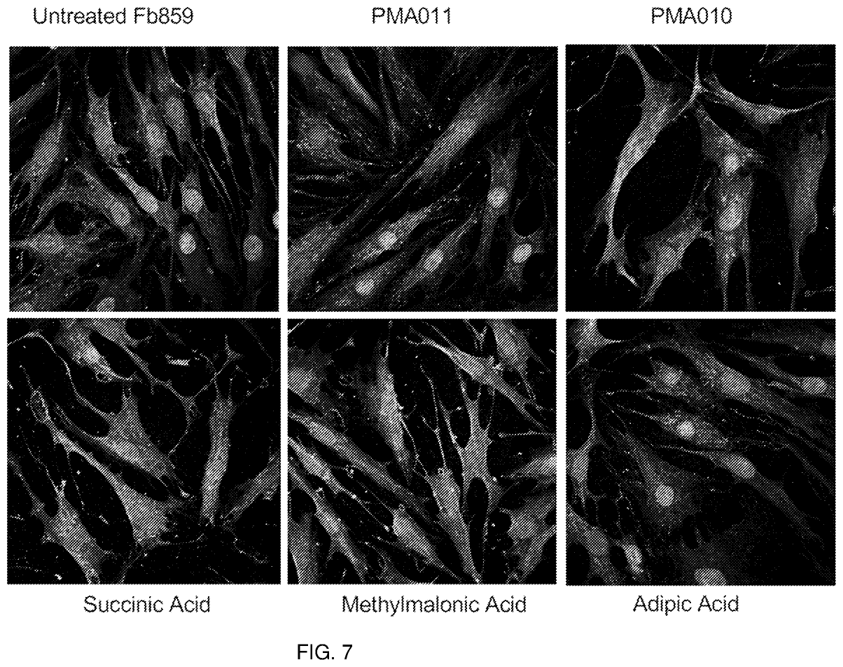

Restoring lysine succinylation: FIG. 7 shows a comparison of lysine succinylation of cellular proteins visualized by immunofluorescence confocal microscopy in untreated and treated PA patient (Fb859) treated with PMA010 and PMA011 or individual components of these two compounds including adipic acid and methylmalonic acid compared to succinic acid. There is an obvious increase in the green staining in all the treatments compared to untreated cells.

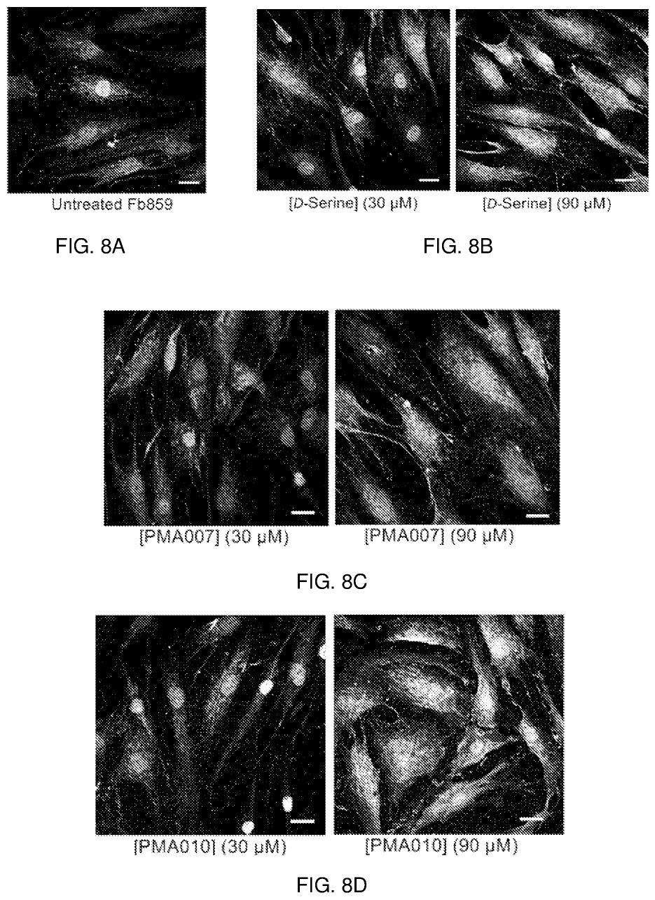

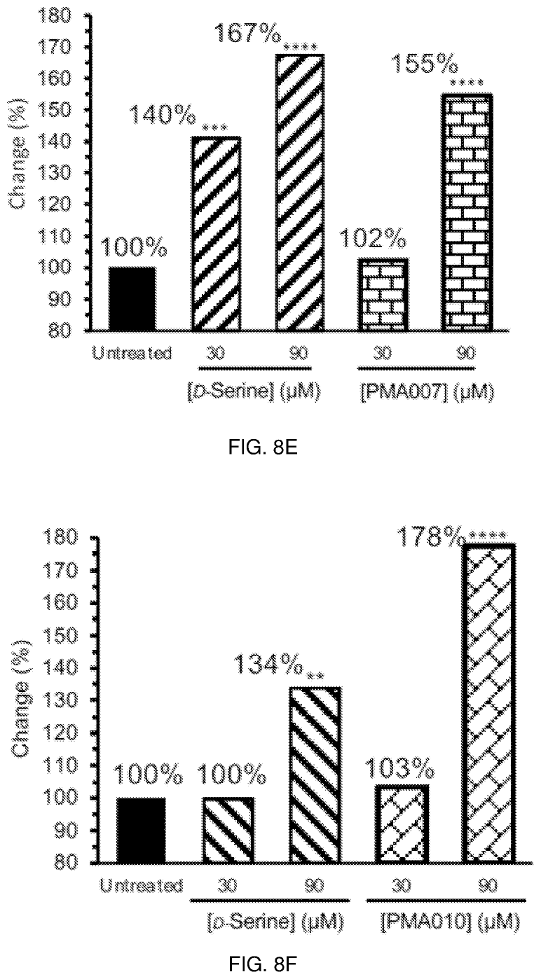

FIGS. 8A-8F are a comparison of lysine succinylation of cellular proteins visualized by immunofluorescence confocal microscopy in untreated and treated PA patient (Fb859). Cells were grown in media without glucose, pyruvate, and glutamine and with stripped FBS. Restoration of lysine succinylation (green) signal in the presence of D-serine is shown in FIG. 8B and is compared to untreated Fb859 in FIG. 8A. Restoration of lysine succinylation (green) signal in the presence of PMA007 at 30 μM and 90 μM is shown in FIG. 8C. Restoration of lysine succinylation (green) signal in the presence of PMA010 at 30 μM and 90 μM is shown in FIG. 8D. Yellow color indicated co-localization of lysine succinylated proteins and the mitochondrial MTCO1 subunit stained with anti-MTCO1 (Red) antibody. DAPI (Blue) included as counterstain. The D-serine and PMA007 treatment data in the bar graph of FIG. 8E are from the quantitation of the images of FIGS. 8A and 8B. The D-serine and PMA010 treatment data in the bar graph of FIG. 8E are from the quantification of images collected from D-serine repeat treatment experiment images and the images collected from FIG. 8D from PMA010 treatment.

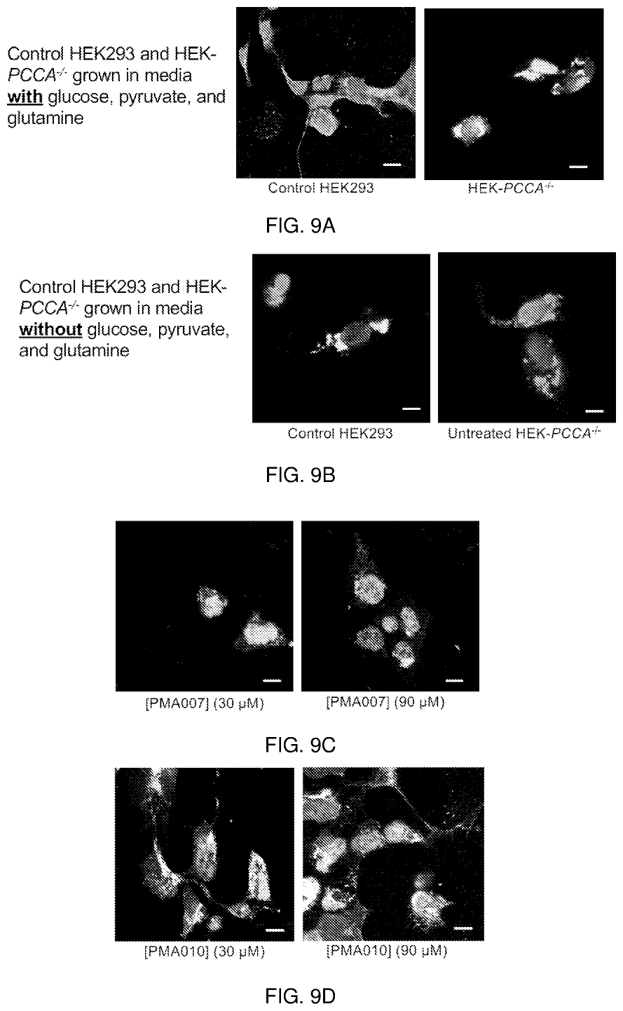

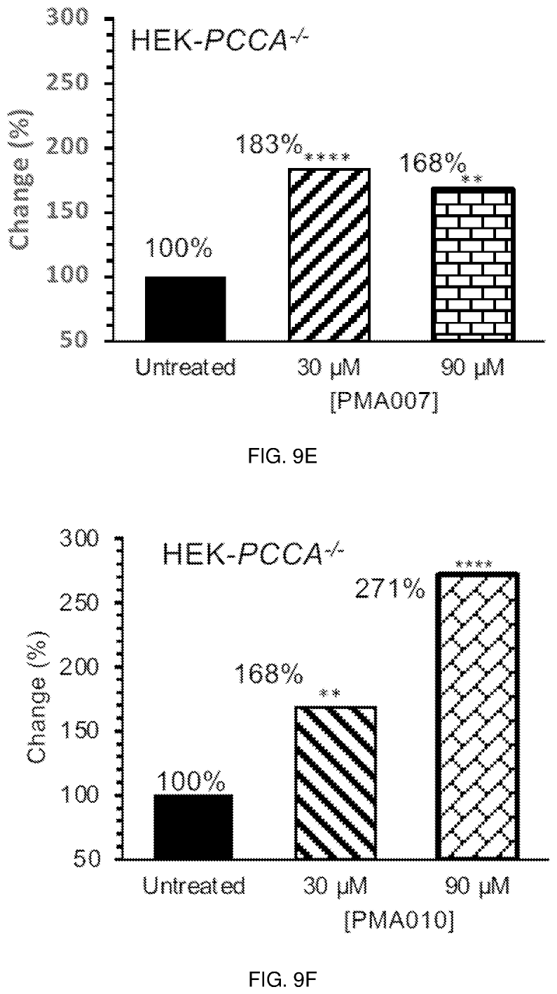

FIGS. 9A-9F are a comparison of lysine succinylation of cellular proteins visualized by immunofluorescence confocal microscopy in human HEK293 control and in untreated and treated HEK293 PCCA^{-/-} knock out cells. Cells were grown in media without glucose, pyruvate, and glutamine in FIGS. 9B, 9C, and 9D. FIG. 9B shows that the substantial loss of lysine succinylation (green) signal is observed in both control and knockout cells in the absence of glucose, pyruvate, and glutamine. However, treatment of the PCCA^{-/-} cells with PMA007 at 30 μM, and at 90 μM (FIG. 9C), or treatment with PMA010 at 30 μM and at 90 μM (FIG. 9D) green color was restored indicating restoration of lysine succinylation. Yellow color indicated co-localization of lysine succinylated proteins and the mitochondrial MTCO1 subunit stained with anti-MTCO1 (Red) antibody. DAPI (Blue) was included as counterstain. The PMA007 treatment data in the bar graph of FIG. 9E are the quantitation of the images of FIG. 9C compared to untreated control in FIG. 9B. The PMA010 treatment data in the bar graph of FIG. 9F are the quantification of images of the images of FIG. 9D compared to untreated control in FIG. 9B.

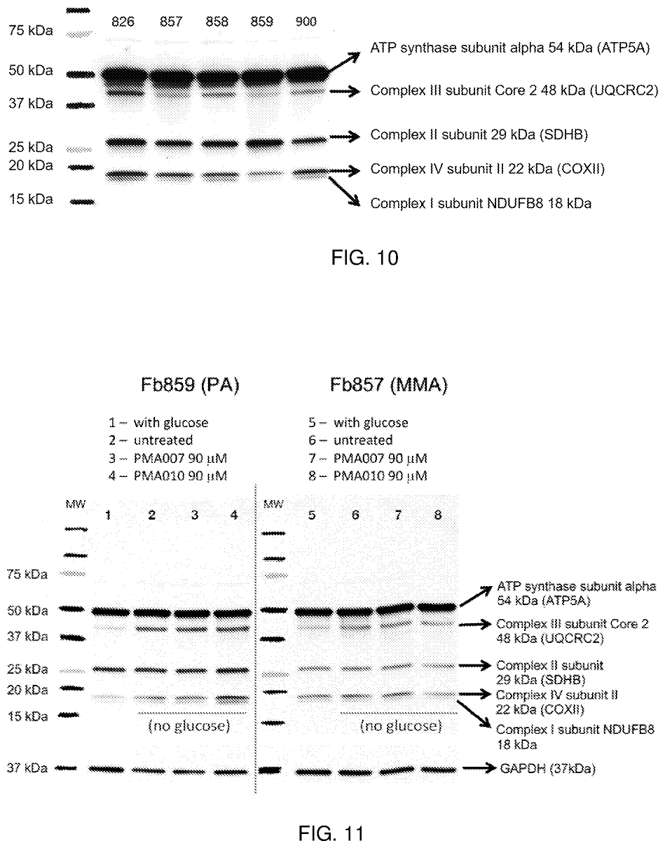

ETC subunits deficiency in PA and MMA: FIG. 10. Shows the control Fb826 control normal levels and the reduction in the signals of various ETC components in PA and MMA cells. Specifically Complex III subunit Core 2 48-kDa (UQCRC2), Complex IV subunit II 22-kDa (COXII) (worst in Fb959), and Complex I subunit NDUFB8 18-kDa. The intensities of the bands for the above ETC subunits were collectively used as biomarkers and changes were observed with the treatments, see below.

FIG. 11, shows correction in the signals of Complex III subunit Core 2 48-kDa (UQCRC2), Complex IV subunit II 22-kDa (COXII), and Complex I subunit NDUFB8 18-kDa that is most pronounced in the propionic acidemia (PA) cell line Fb859 compared to the methylmalonic aciduria (MMA) cell line Fb857. PMA010 appears to have a better effect compared to PMA007. The Complex II subunit Core 2 (UQCRC2) was improved in the untreated cells (no glucose, pyruvate, or fat in medium).

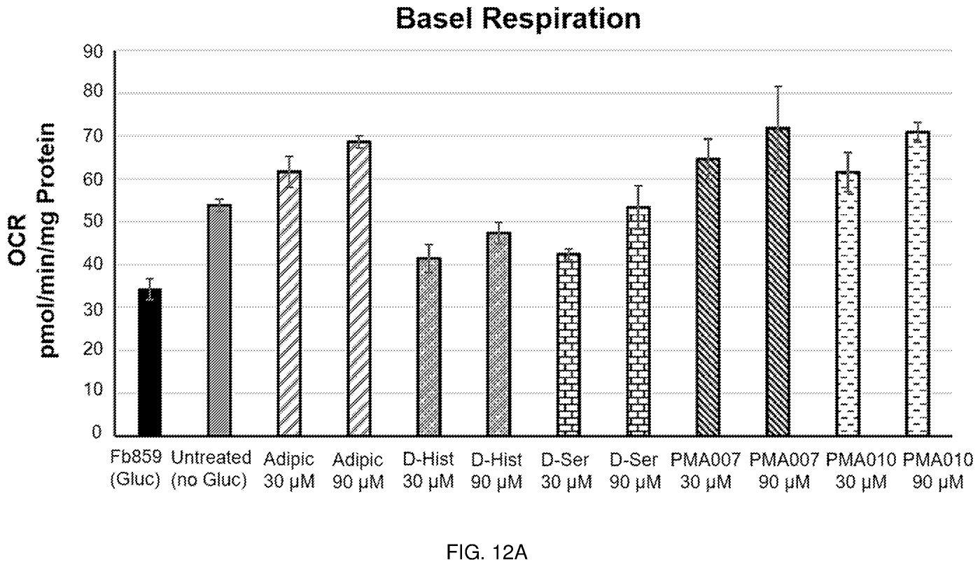

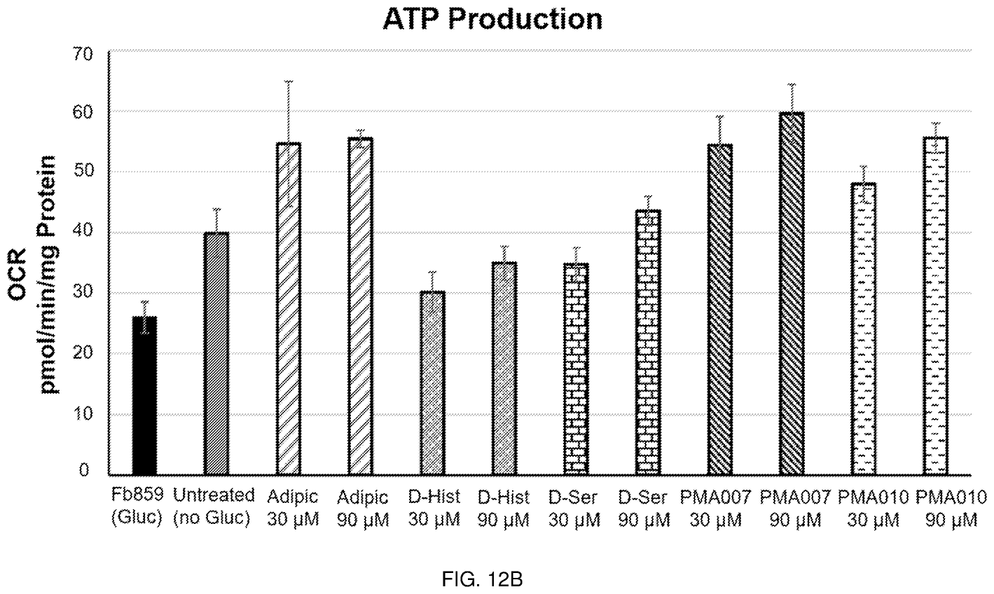

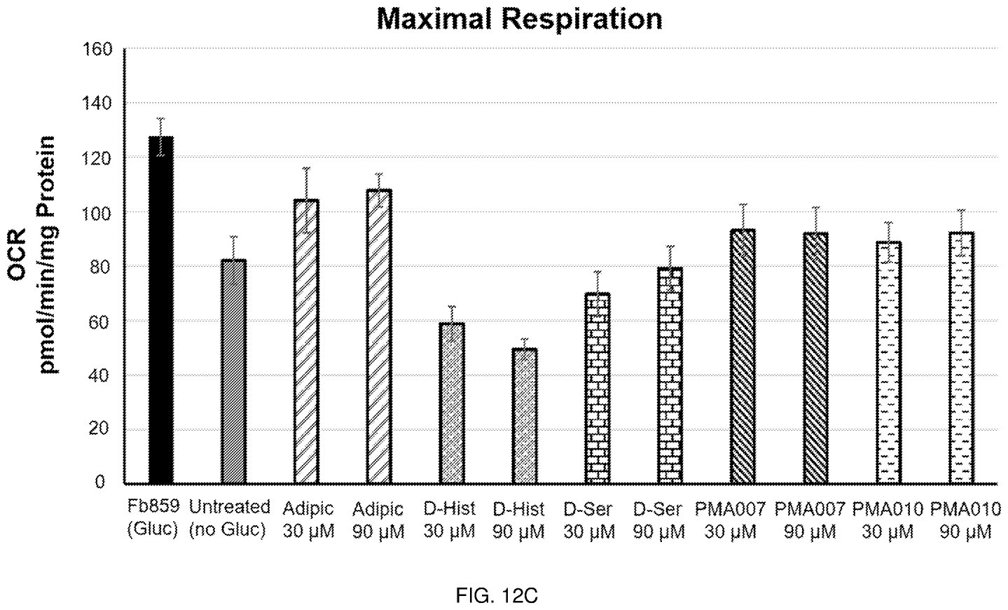

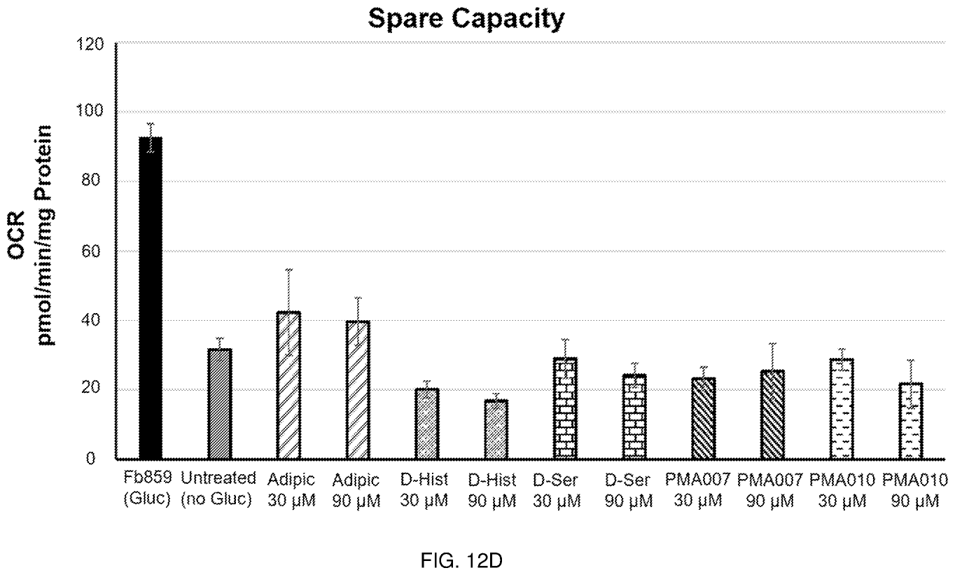

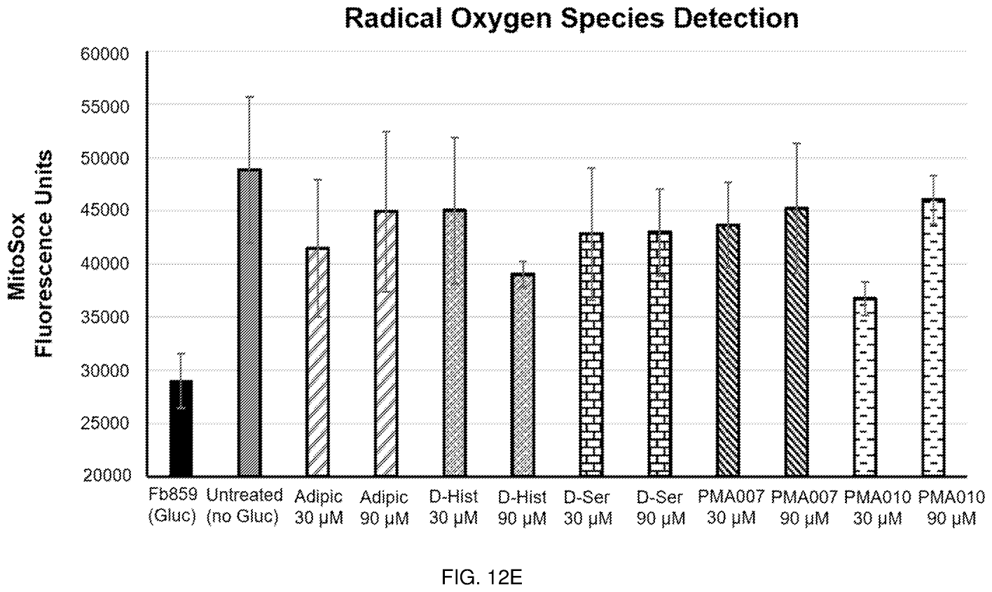

O₂ consumption parameters: FIGS. 12A-12F, Oxygen consumption rate changes with addition of various inhibitors of the ETC in response to treatment of PA patient cells, Fb859, with D-serine, D-histidine, adipic acid, PMA007, and PMA010 were compared to the response to the treatment of individual components at 30 μM and 90 μM for 72 h. Data are reported as means±SD. Replicates n=5-8. Data were tested for normal distribution by the Shapiro-Wilk test. Variances were homogeneous (p>0.05) and the data followed a normal distribution (p>0.05). *P<0.05, **P<0.01, ***P<0.001 (one-way analysis of variance (ANOVA) followed by the post-hoc Tukey multiple range test when F was significant). ns: not significant. Overall, the oxygen consumption parameters were improved in the presence of the compounds tested when compared to the individual components, D-Serine and D-Histidine, except adipic acid. More detailed studies are necessary to decern the importance of the contribution of the D-amino acid components of the PMA family of compounds in replenishing the Krebs cycle intermediates of compounds compared to serving as carrier for the dicarboxylic acid component.

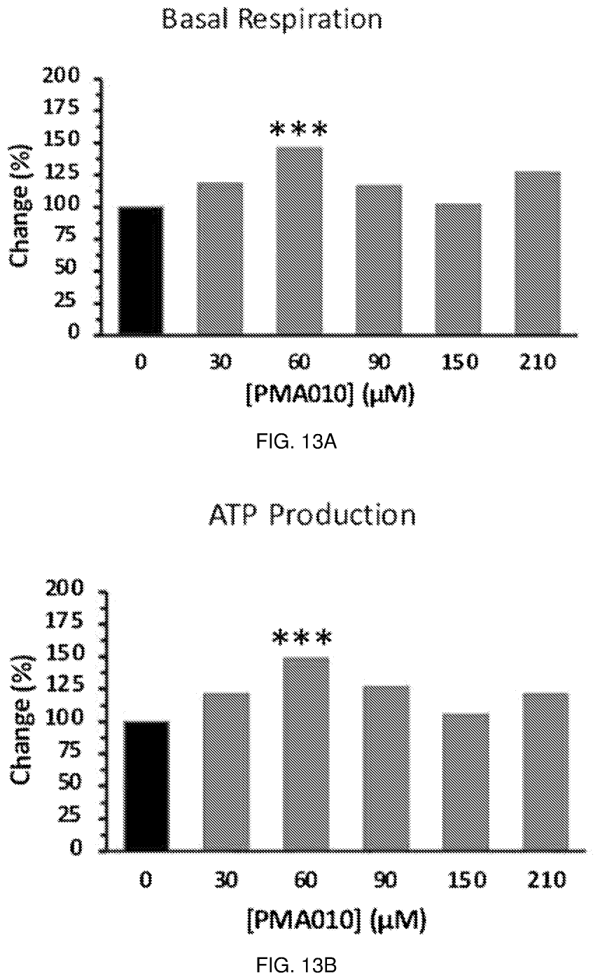

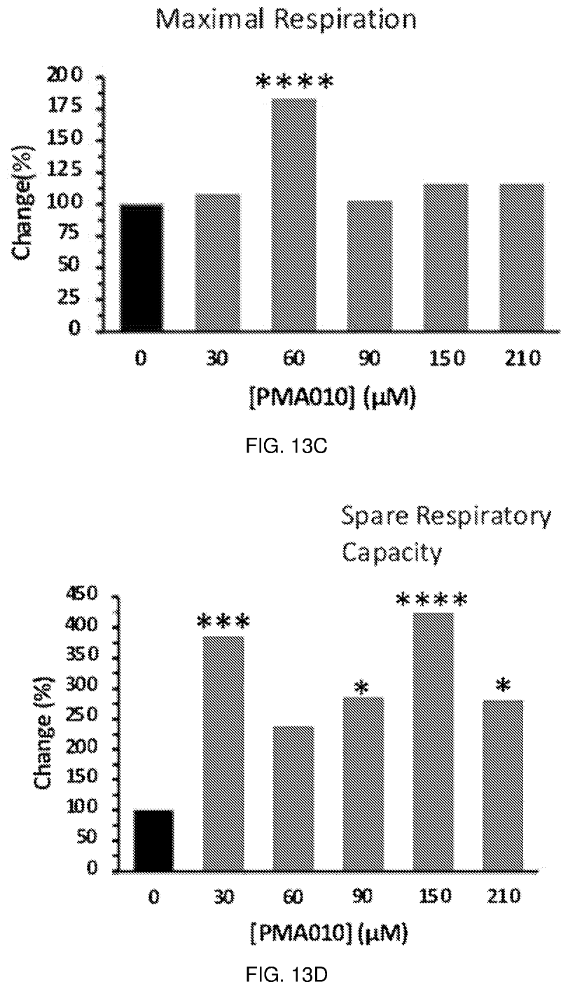

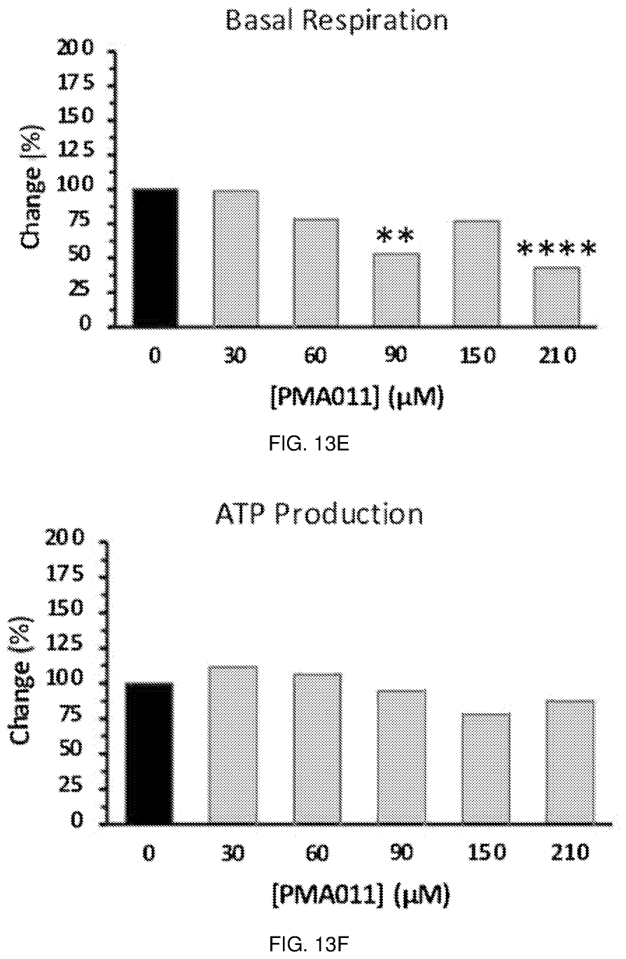

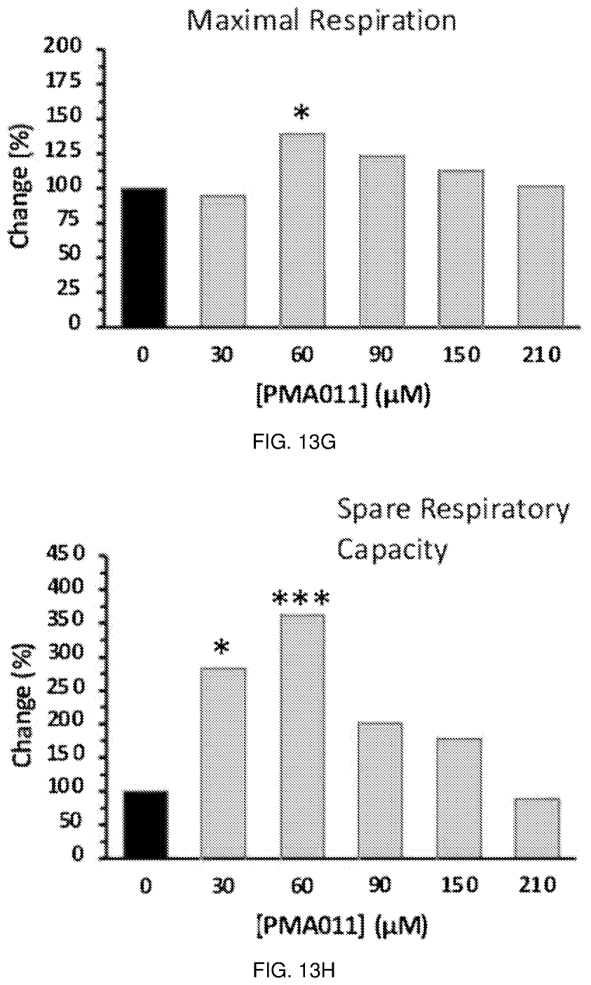

O₂ consumption parameters: FIGS. 13A-13H, Oxygen consumption rate changes with addition of various inhibitors in response to treatment of PA patient cells, Fb859, with PMA010 (FIGS. 13A-13D) and PMA011 (FIGS. 13E-13J). Data in these figures are presented as percentages of changes to the untreated cells. While basal respiration may represent oxygen consumption at resting state and therefore is a function of cell stress, the significant of the change in spare capacity, as high as 3-4 times the untreated, shows the effectiveness of these two compounds in improving the bioenergetics of the cells.

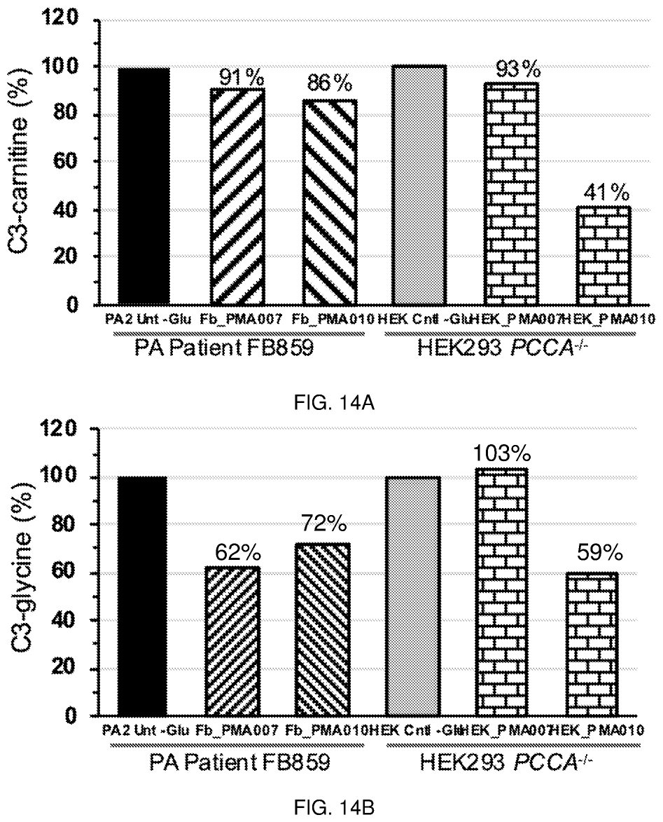

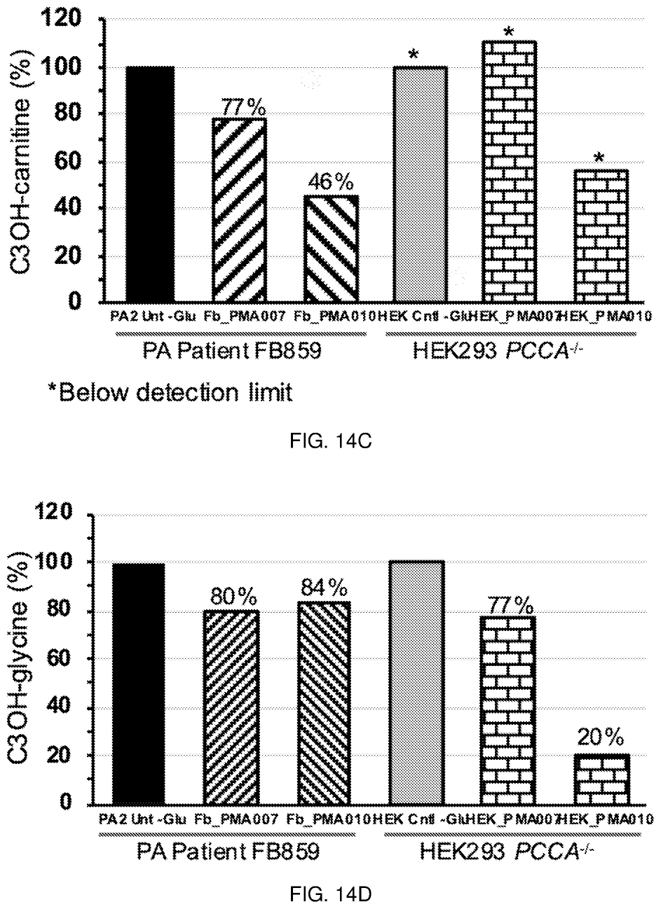

Acylcarntine evidence: FIGS. 14A-14D, Quantitation of propionylcarnitine (FIG. 14A), propionylglycine (FIG. 14B), hydroxypropionylcarnitine (FIG. 14C), and hydroxypropionyl glycine (FIG. 14D) in media of Fb859 cells and HEK293 PCCA^{-/-} cells containing PMA007 or PMA010. The decrease detected across the propionyl-CoA alternative metabolites is significant as a primary biomarker of the disease. Excess propionyl-CoA thiolysis to the acid converts to the four metabolites mostly in liver with carnitine and glycine being the detoxifying agents. PMA010 seem to be more effective in HEK293 PCCA^{-/-} cells causing reduction in propionylcarnitine (decreased by 59%), propionylglycine (decreased by 41%), and hydroxypropionyl glycine (decreased by 80%). Media samples were processed as indicated in Materials and Procedures, below, using mass spectroscopy.

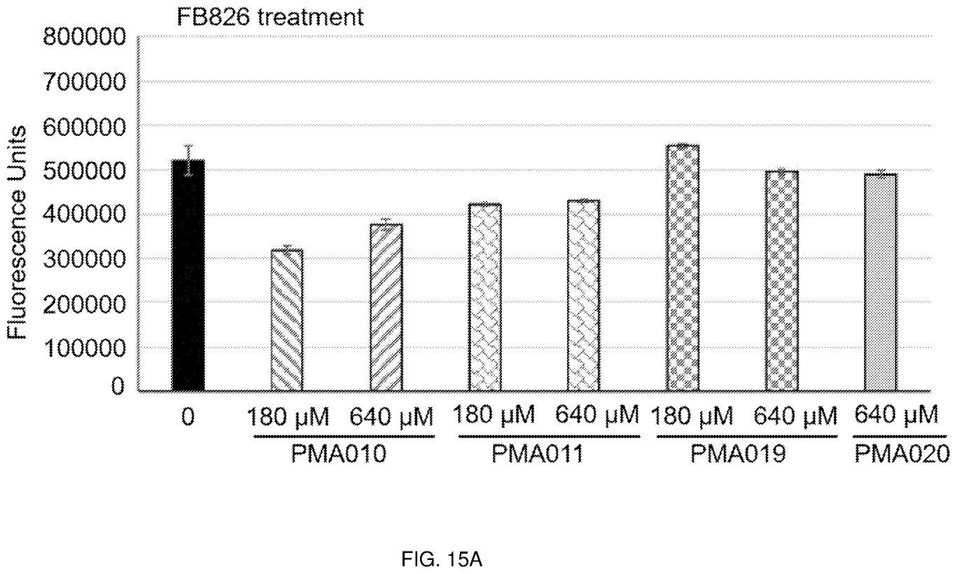

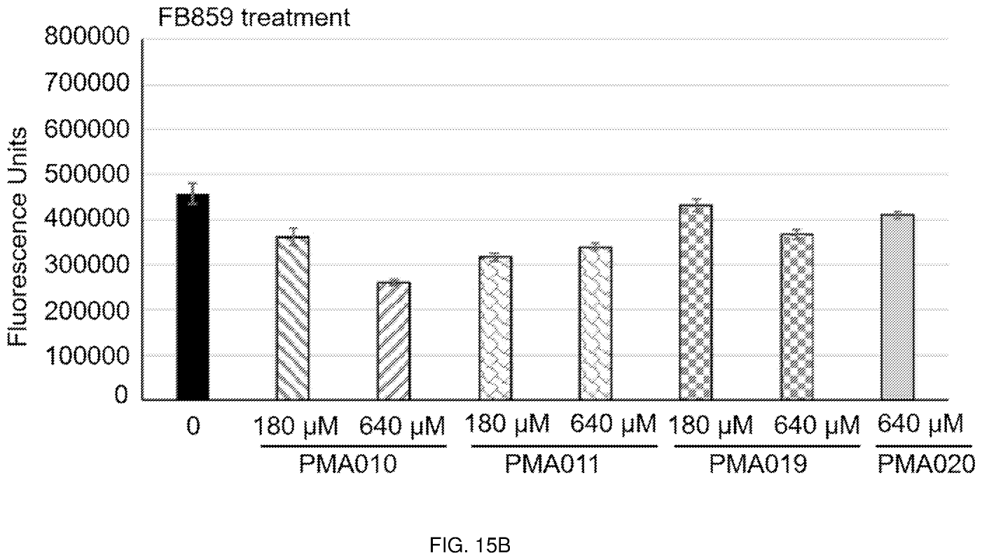

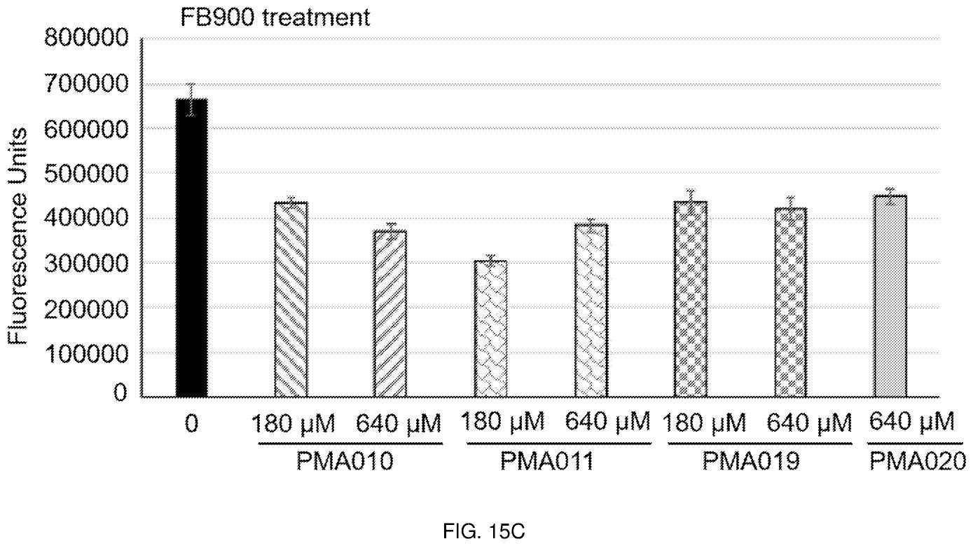

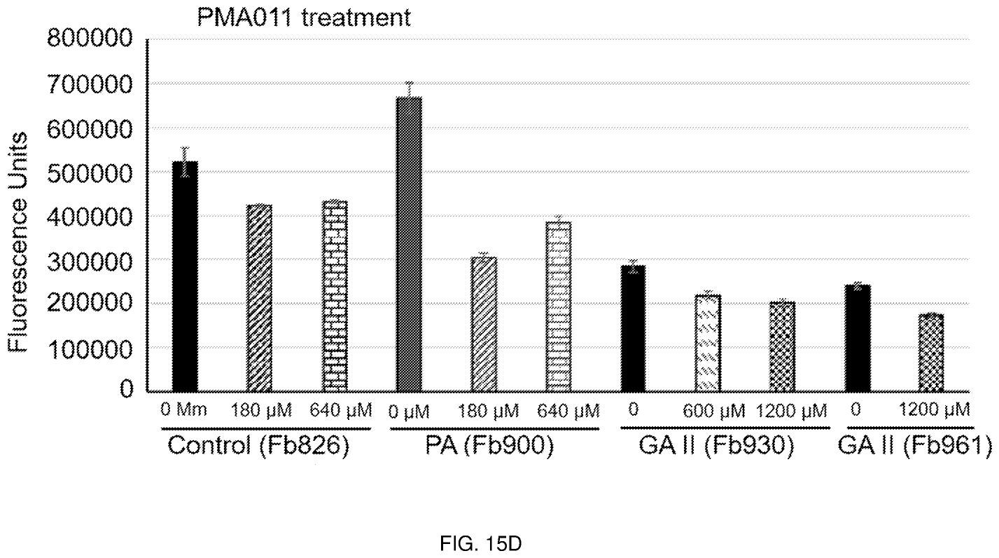

Evidence for the effect of PMA compounds in decreasing detectable reactive oxygen species (ROS) in PA and GA II cells: FIGS. 15A-15D show the level of ROS, a biomarker associated with occurrence of cellular stress in the untreated PA cells and the effect of treatment with various amounts of PMA010, PMA011, PMA019, and PMA020 on ROS generation in Control FB826 (FIG. 15A), FB859 PA cells (FIG. 15B), and FB900 PA cells (FIG. 15C). PMA010 caused significant decrease in ROS detected in Control cells (FB826) (FIG. 15A) and in FB900 (FIG. 15C), another PA cell line, and PMA011 was similarly effective in FB900 (FIG. 15C). Preliminary test of the effect of PMA011 on GA II (glutaric acidemia II) FB930 and FB961 cell lines also show significant decrease in ROS when treated compared to no treatment (FIG. 15D).

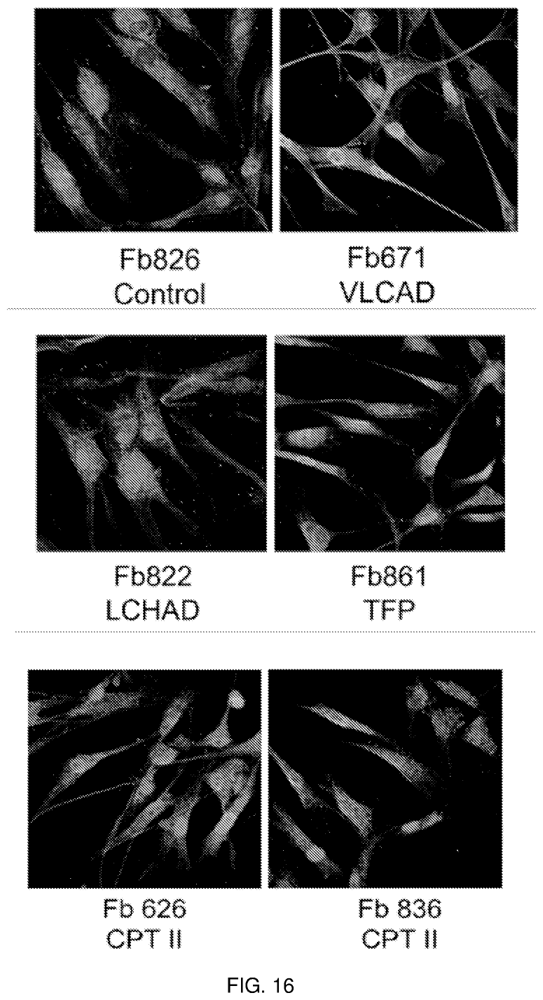

Evidence for reduced lysine succinylation in fatty acid β-oxidation disorders: FIG. 16 shows the significant reduction in antigenic signal for succinyllysine (green stain), severe lysine hyposuccinylation. in fatty acid oxidation disorders as indicated. Notable with the Control normal cell line the yellow color indicates the overlap of the signal for succinyllysine (green) and Anti-MTCO1 (mitochondrial marker, red). In the TFP, CPTII and VLCAD cell lines the red is dominant indicating reduced lysine succinylation.

CONCLUSION: The results are all consistent and unequivocal pointing to the effectiveness of the three compounds for the PA cells (propionyl-CoA carboxylase deficient) on the molecular level in terms of being rich sources of succinyl-CoA, albeit PMA010 show slightly better performance in the in vitro model. While the media propionylcarnitine seems modestly decreased at 15%, however, propionylcarnitine was artificially high to begin with due to the forced use of amino acids for energy as cells were deprived of glucose, pyruvate, or fatty acids under the used experimental conditions. In MMA the performance of the compounds is not as notable and is hypothesized due to methylmaloyl-CoA competing to modify the same reactive lysine sites that succinyl-CoA targets. This suggests that PCC inhibitors can act synergistically with PMA010 to remedy the deficiency of succinyl-CoA while PCC inhibitors limiting the buildup of methylmaloyl-CoA.

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

1. A method of providing an anaplerotic compound to a patient in need therefor, comprising administering to the patient a compound having the structure A-B or B-A′, where:
A comprises a dicarboxylate, a tricarboxylic, or a straight or branched-chain fatty acid moiety selected from: an adipic acid, a 3-hydroxy-adipic acid, a 3-ketoadipic acid, a glutaric acid, a succinic acid, methylmalonic acid, a citric acid, an isocitric acid, α-ketoglutaric acid, an oxaloacetic acid, a heptanoic acid, a 2,6-dimethylheptanoic acid, a 4,8-dimethylnonanoic acid, a 6-amino-2,4-dimethylheptanoic acid, a straight chain fatty acid, or a branched-chain fatty acid;
A′ comprises a straight or branched-chain fatty acid moiety selected from: 6-hydroxy-hexanoic acid, 6-amino-hexanoic acid, or 6-amino-2,4-dimethylheptanoic acid and
B is an amino acid, D-dipeptide, or D-tripeptide of D-amino acids, comprising at least one amino acid selected from D-Ser, D-Ala, D-His, D-Lys, D-Glu, D-Gln, D-Asp, D-Asn, D-Leu, D-Ile, D-Thr, or D-Val linked to A by an amide bond or by an amide bond or ester bond in the case of D-Ser, or to A′ by an ester bond or an amide bond,
or a pharmaceutically acceptable salt thereof.

2. The method of claim 1, wherein the compound has the structure A-B, where:
A comprises an adipic acid moiety or a methylmalonic acid moiety; and
B comprises from 1-3 D-amino acids selected from D-Ser, D-Ala, D-Lys, D-Leu, or D-His linked to A by an amide bond or by an amide bond or an ester bond in the case of D-Ser, or a pharmaceutically acceptable salt thereof for the treatment of fatty acid oxidation disorders.

3. The method of claim 1, wherein the anaplerotic compound enters the Krebs cycle as an intermediate.

4. A method of treating a patient having a propionic acidemia (PA), a methymalonic aciduria (MMA), or a fatty acid oxidation disorder, and having abnormally low amounts of anaplerotic intermediates, comprising administering to the patient a compound having the structure A-B or B-A′, wherein:
a. for treating PA, A comprises a dicarboxylate, a tricarboxylic, or an even straight-chain fatty acid moiety selected from: an adipic acid, a glutaric acid, a succinic acid, methylmalonic acid, a citric acid, an isocitric acid, an oxaloacetic acid, α-ketoglutaric acid, A′ comprises a straight even-chain dicarboxylic or fatty acid moiety selected from: a 6-hydroxy-hexanoic acid, a 6-amino-hexanoic acid, and B is a D-amino acid, D-dipeptide, or D-tripeptide of D-amino acids comprising at least one amino acid selected from D-Ser, D-Ala, D-His, D-Lys, D-Glu, D-Gln, D-Asp, D-Asn, or D-Leu linked to A by an amide bond or by an amide bond or ester bond in the case of D-Ser or to A′ by an amide bond or an ester bond, or a pharmaceutically acceptable salt thereof;
b. for treating MMA, A comprises a dicarboxylate, a tricarboxylic, or an even straight-chain fatty acid moiety selected from: an adipic acid, a glutaric acid, a succinic acid, a citric acid, an isocitric acid, an oxaloacetic acid, and α-ketoglutaric acid; and A′ comprises a straight even-chain dicarboxylic or fatty acid moiety selected from: a 6-hydroxy-hexanoic acid or a 6-amino-hexanoic acid; and B is a D-amino acid, D-dipeptide, or D-tripeptide of D-amino acids, comprising at least one amino acid selected from D-Ser, D-Ala, D-His, D-Lys, D-Glu, D-Gln, D-Asp, D-Asn, or D-Leu linked to A by an amide bond or by an amide bond or ester bond in the case of D-Ser or to A′by an amide bond or an ester bond, or a pharmaceutically acceptable salt thereof; or
c. for treating a fatty acid oxidation disorder, A comprises a dicarboxylate, a tricarboxylic, or an even straight-chain or branched-chain fatty acid moiety selected from: an adipic acid, a glutaric acid, a succinic acid, methylmalonic acid, a citric acid, an isocitric acid, an oxaloacetic acid, α-ketoglutaric acid, a heptanoic acid, a 2,6-dimethylheptanoic acid, a 4,8-dimethylnonanoic acid, a 6-amino-2,4-dimethylheptanoic acid, an even straight chain fatty acid, or a branched-chain fatty acid; A′ comprises a straight even-chain dicarboxylic or fatty acid moiety selected from: 6-hydroxy-hexanoic acid, 6-amino-hexanoic acid, or 6-amino-2,4-dimethylheptanoic acid; and B is a D-amino acid, D-dipeptide, or D-tripeptide of D-amino acids, comprising at least one amino acid selected from D-Ser, D-Ala, D-His, D-Lys, D-Glu, D-Gln, D-Thr, D-Ile, D-Val, D-Asp, D-Asn, or D-Leu linked to A by an amide bond or by an amide bond or ester bond in the case of D-Ser or to A′ by an amide bond or an ester bond, or a pharmaceutically acceptable salt thereof.

5. The method of claim 4, wherein the compound has the structure A-B, where:
A comprises an adipic acid moiety or a methylmalonic acid moiety; and
B is a D-amino acid, D-dipeptide, or D-tripeptide of D-amino acids, comprising at least one amino acid selected from D-Ser, D-Ala, D-Lys, D-Leu, and D-His, linked to A by an amide bond or by an amide bond or an ester bond in the case of D-Ser, or a pharmaceutically acceptable salt thereof, in an amount effective to normalize levels of the anaplerotic intermediates.

6. The method of claim 1, wherein
the patient has propionic acidemia (PA); methylmalonic aciduria (MMA) transient, due to transcobalamin receptor defect; methylmalonic aciduria, cblA type; methylmalonic aciduria, cblB type; medium chain acyl-CoA dehydrogenase (MCAD) deficiency; very long chain acyl-CoA dehydrogenase (VLCAD) deficiency; trifunctional protein (TFP) deficiency; long chain 3-hydroxyacyl-CoA dehydrogenase (LCHAD) deficiency; carnitine palmitoyl transferase II (CPT II) deficiency; glutaric acidemia I (GA I or GA1); or glutaric acidemia II (GA I or GA2).

7. (canceled)

8. The method of claim 1, wherein the patient has reduced succinyl-CoA or lysine succinylation.

9. The method of claim 1, wherein the patient has glutaric acidemia I (GA I or GA1) with the exclusion of glutaric acid or lysine in the compound structure, or glutaric acidemia II (GA II or GA 2) with the exclusion of glutaric acid or lysine, fatty acids and branched chain amino acids that require ETF or ETF dehydrogenase for their breakdown in the compound structure.

10. The method of claim 1, wherein B comprises one or more of D-Ser, D-Ala, D-Lys, D-Leu, and/or D-His.

11. The method of claim 1, wherein B is chosen from: -D-Ser, -D-Ala, -D-His, -D-His-D-Ala-D-Ser, -D-His-D-Ser-D-Ala, -D-Ser-D-His-D-Ala, -D-Ser-D-Ala-D-His, -D-Ala-D-Ser-D-His, -D-Ala-D-His-D-Ser, -D-Ala-D-His-D-Ser, -D-His-D-Ala, -D-His-D-Ser, -D-Ser-D-His, -D-Ala-D-Ser, or -D-Ala-D-His, wherein D-Ser is linked by an amide bond (—N-linked) or an ester bond (—O-linked).

12. The method of claim 1, wherein the compound has the structure: 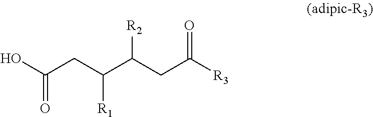
where R₁ and R₂ are, independently H, hydroxyl, or oxygen connected by a double bond to form a keto moiety, where at least one of R₁ and R₂ is H, and R₃ is linked via an ester bond or an amide bond, and comprises from 1-3 D amino acids selected from D-Ser, D-Ala, D-His, D-Lys, D-Glu, D-Gln, D-Asp, D-Asn, D-Leu, and D-Val, or is one of: -D-Ser, —O-D-Ser, -D-Ala, -D-His, -D-His-D-Ala-D-Ser, -D-His-D-Ala-O-D-Ser, -D-His-D-Ser-D-Ala, -D-His-O-D-Ser-D-Ala, -D-Ser-D-His-D-Ala, —O-D-Ser-D-His-D-Ala, -D-Ser-D-Ala-D-His, —O-D-Ser-D-Ala-D-His, -D-Ala-D-Ser-D-His, -D-Ala-O-D-Ser-D-His, -D-Ala-D-His-D-Ser, -D-Ala-D-His-D-Ser, -D-His-D-Ala, -D-His-D-Ser, -D-His-O-D-Ser, -D-Ser-D-His, -D-Ala-O-D-Ser, or -D-Ala-D-His.

13. The method of claim 1, wherein the compound has the structure: 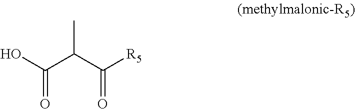
where R₅ is linked via an ester or an amide bond, and comprises from 1-3 D amino acids selected from D-Ser, D-Ala, D-His, D-Lys, D-Glu, D-Gln, D-Asp, D-Asn, D-Leu, and D-Val, or is one of: -D-Ser, —O-D-Ser, -D-Ala, -D-His, -D-His-D-Ala-D-Ser, -D-His-D-Ala-O-D-Ser, -D-His-D-Ser-D-Ala, -D-His-O-D-Ser-D-Ala, -D-Ser-D-His-D-Ala, —O-D-Ser-D-His-D-Ala, -D-Ser-D-Ala-D-His, —O-D-Ser-D-Ala-D-His, -D-Ala-D-Ser-D-His, -D-Ala-O-D-Ser-D-His, -D-Ala-D-His-D-Ser, -D-Ala-D-His-D-Ser, -D-His-D-Ala, -D-His-D-Ser, -D-His-O-D-Ser, -D-Ser-D-His, -D-Ala-O-D-Ser, -D-Ala-D-His, -D-Leu-D-His, or -D-Lys-D-His.

14. The method of claim 1, wherein the compound is chosen from: 4-hydroxyladipic acid-O-D-Ser; 4-hydroxyladipic acid-O-D-Ser-D-Ala; 4-hydroxyladipic acid-O-D-Ser-D-Ala; 2-ketoglutaric acid-D-2-hydroxyglycine-D-Ala; 2-ketoglutaric acid-O-D-Ser-D-Ala; 4-ketoadipic acid-O-L-Ser; 4-ketoadipic acid-O-D-Ser-D-His-succinyl CoA; 2-ketoglutaric acid-O-D-Ser-D-His; 2-ketoglutaric acid-D-Ala-D-His; 4-ketoadipic acid-D-Ala-D-His; methylmalonic acid-O-D-Ser-D-His; methylmalonic acid-D-Ala-D-His; adipic acid-D-Ala-D-Ser-D-His; adipic acid-D-Ser-D-His; methylmalonic acid-D-Leu-D-His; and methylmalonic acid-D-Lys-D-His.

15-21. (canceled)

22. The method of claim 1, wherein the patient has propionic acidemia or methylmalonic aciduria, and the method further comprises administering to the patient an inhibitor that blocks the formation of the propionoyl-CoA or methylmalonoyl-CoA

23. The method of claim 22, wherein the inhibitor that blocks the formation of the propionoyl-CoA acid or methylmalonoyl-CoA is 2,2-dimethybutyric acid or sodium 2,2-dimethylbutanoate.

24. The method of claim 1, further comprising administering to the patient an inhibitor that restricts propionyl-CoA or methylmalonyl-CoA production from various sources.

25. (canceled)

26. A compound having the structure A-B or B-A′, wherein:
A comprises a dicarboxylate, a tricarboxylic, or a straight or branched-chain fatty acid moiety selected from: an adipic acid, a 3-hydroxy-adipic acid, a 3-ketoadipic acid, a glutaric acid, a succinic acid, methylmalonic acid, a citric acid, an isocitric acid, α-ketoglutaric acid, an oxaloacetic acid, a heptanoic acid, a 2,6-dimethylheptanoic acid, a 4,8-dimethylnonanoic acid, a 6-amino-2,4-dimethylheptanoic acid, a straight chain fatty acid, or a branched-chain fatty acid;
A′ comprises a straight or branched-chain fatty acid moiety selected from: 6-hydroxy-hexanoic acid (hydroxyl linked to B carboxylate as an ester bond), 6-amino-hexanoic acid, or 6-amino-2,4-dimethylheptanoic acid; and
B is an amino acid, D-dipeptide, or D-tripeptide of amino acids, comprising at least one amino acid selected from D-Ser, D-Ala, D-His, D-Lys, D-Glu, D-Gln, D-Asp, D-Asn, D-Leu, D-Ile, D-Thr, or D-Val linked to A by an amide bond or by an amide bond or ester bond in the case of D-Ser, or to A′ by an ester bond or an amide bond,
or a pharmaceutically acceptable salt thereof.

27. The compound of claim 26, wherein the compound has the structure A-B, where:
A comprises an adipic acid moiety or a methylmalonic acid moiety; and
B is a D-amino acid, D-dipeptide, or D-tripeptide of D-amino acids, comprising at least one amino acid selected from D-Ser, D-Ala, D-Lys, D-Leu, and D-His, linked to A by an amide bond or by an amide bond or an ester bond in the case of D-Ser, or a pharmaceutically acceptable salt thereof.

28-29. (canceled)

30. The compound of claim 26, having the structure: 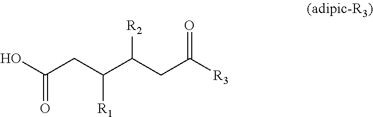
where R₁ and R₂ are, independently H, hydroxyl, or oxygen connected by a double bond to form a keto moiety, where at least one of R₁ and R₂ is H, and R₃ is linked via an ester (—O-linked) or amide (—N-linked) bond, and comprises from 1-3 D amino acids selected from D-Ser, D-Ala, D-His, D-Lys, D-Glu, D-Gln, D-Asp, D-Asn, D-Leu, and D-Val or is one of: -D-Ser, —O-D-Ser, -D-Ala, -D-His, -D-His-D-Ala-D-Ser, -D-His-D-Ala-O-D-Ser, -D-His-D-Ser-D-Ala, -D-His-O-D-Ser-D-Ala, -D-Ser-D-His-D-Ala, —O-D-Ser-D-His-D-Ala, -D-Ser-D-Ala-D-His, —O-D-Ser-D-Ala-D-His, -D-Ala-D-Ser-D-His, -D-Ala-O-D-Ser-D-His, -D-Ala-D-His-D-Ser, -D-Ala-D-His-D-Ser, -D-His-D-Ala, -D-His-D-Ser, -D-His-O-D-Ser, -D-Ser-D-His, -D-Ala-O-D-Ser, or -D-Ala-D-His.

31. The compound of claim 26, having the structure: 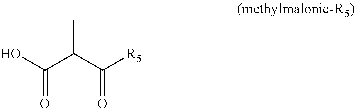
where R₅ is linked via an ester (—O-linked) or amide (—N-linked) bond, and comprises from 1-3 D amino acids selected from D-Ser, —O-D-Ser, D-Ala, D-His, D-Lys, D-Glu, D-Gln, D-Asp, D-Asn, D-Leu, and D-Val, or is one of: -D-Ser, —O-D-Ser, -D-Ala,-D-His, -D-His-D-Ala-D-Ser, -D-His-D-Ala-O-D-Ser, -D-His-D-Ser-D-Ala, -D-His-O-D-Ser-D-Ala, -D-Ser-D-His-D-Ala, —O-D-Ser-D-His-D-Ala, -D-Ser-D-Ala-D-His, —O-D-Ser-D-Ala-D-His, -D-Ala-D-Ser-D-His, -D-Ala-O-D-Ser-D-His, -D-Ala-D-His-D-Ser, -D-Ala-D-His-D-Ser, -D-His-D-Ala, -D-His-D-Ser, -D-His-O-D-Ser, -D-Ser-D-His, —O-D-Ser-D-His-D-Ser-D-Ala, —O-D-Ser-D-Ala-D-Ala-D-Ser, -D-Ala-O-D-Ser, -D-Ala-D-His, -D-Leu-D-His, or -D-Lys-D-His.

32. (canceled)
